(19) United States
(12) Patent Application Publication
(10) Pub. No.: US 2019/0244697 A1
(43) Pub. Date: Aug. 8, 2019

(54) INFRASTRUCTURE FOR UTILIZING ELECTRONIC RECORDS

(71) Applicant: Thomas Farrell, Doylestown, PA (US)

(72) Inventor: Thomas Farrell, Doylestown, PA (US)

(21) Appl. No.: 16/267,109

(22) Filed: Feb. 4, 2019

Related U.S. Application Data
(60) Provisional application No. 62/625,795, filed on Feb. 2, 2018.

(51) Int. Cl. G16H 20/10; G16H 10/60

(52) U.S. Cl. CPC G16H 20/10; G16H 10/60

(57) ABSTRACT

The disclosure according to some exemplary embodiments relates generally to an infrastructure for electronic records (e.g., electronic medical records (EMRs), electronic health records (EHRs), and personal health records (PHRs)). The infrastructure allows the use, for example, of the electronic records and other information sources to determine drug and other treatment prices that may differ from listed prices based on various factors, such as approved indication treated, the organization and/or individual responsible for payment, whether and what other drug or drugs are used as part of the treatment regimen, a diagnosis and treatment plan, and responses to treatment, among other factors. Advantageously, in some embodiments, systems and methods using the infrastructure allows patients to receive lower drug and treatment prices tailored to the patients' needs and can increase patients' access to drugs and other treatments.

### CROSS REFERENCE TO RELATED APPLICATION

This application claims the benefit of U.S. Provisional Patent Application No. 62/625,795, filed Feb. 2, 2018, which is hereby incorporated by reference here in its entirety.

### TECHNICAL FIELD

Disclosed apparatuses, systems, and methods relate generally to an infrastructure for utilizing electronic records. In some embodiments, the disclosed apparatuses, systems, and methods relate more particularly to an infrastructure for utilizing electronic records of a patient's medical information.

### BACKGROUND

Patient medical, health, and other information may be stored in electronic records such as electronic medical records (EMR), electronic health records (EHR), personal health records (PHR), and other electronic records from payer systems. When a patient visits a healthcare provider to, for example, receive health advice and/or treatment for a disease or condition, the results of the visit, such as test results, treatments administered, diagnoses, prognoses, etc., are often stored in an electronic record such as an EMR. An EMR generally serves as a single healthcare practice's digital version of a patient's chart, and it generally contains the patient's medical history, diagnoses and treatments by a particular healthcare provider. When the patient subsequently receives additional treatment, the EMR is updated with additional treatment information, such as notes by the healthcare provider and information about the patient's responsiveness to treatment. Another type of electronic record is an EHR, which generally provides a more inclusive snapshot of the patient's medical history, such as related to patients' allergies, radiology images, pharmacy record, and laboratory test results both internal and external to the healthcare provider. An EMR generally provides a narrower view of a patient's medical history, while an EHR generally provides a more comprehensive view of the patient's overall health. Information in an EHR is typically entered and accessed by health care providers. A further type of electronic record is a PHR. A PHR may include health information from a variety of sources, including multiple health care providers and the patients themselves. PHRs may be standalone or connected. In standalone PHRs, patients generally fill in information from their own records and memories, and the data is generally stored on the patients' computers or on the internet. Patients can decide whether to share the information with providers, family members, or anyone else involved in their healthcare. In some cases, information can be downloaded from other sources into the PHR. Connected PHRs are generally linked to a specific health care organization's EHR system or a health plan's information system. Patients generally access information through a secure portal. Typically, patients can view information such as laboratory results, immunization history, or due dates for certain health screenings.

Treatment for a patient's disease or condition often includes the patient taking or being administered a drug as part of the treatment regimen. In conventional drug pricing systems, price per unit drug prices are typically set or negotiated independent of factors such as what indication a drug is used to treat for a particular patient and/or a particular patient's response to the treatment. For example, a drug such as TAXOL® (paclitaxel), which is a chemotherapeutic agent approved for treating different types of cancer, has multiple approved indications. TAXOL® Prescribing Information (Rev. April 2011). TAXOL® is indicated as first-line and subsequent therapy for the treatment of advanced carcinoma of the ovary. As first-line therapy, it is indicated in combination with cisplatin. TAXOL® is indicated for the adjuvant treatment of node-positive breast cancer administered sequentially to standard doxorubicin-containing combination chemotherapy. TAXOL® is indicated for the treatment of breast cancer after failure of combination therapy for metastatic disease or relapse within six months of adjuvant chemotherapy. In combination with cisplatin, TAXOL® is indicated for the first-line treatment of non-small cell lung cancer in patients who are not candidates for potentially curative surgery and/or radiation therapy. TAXOL® is also indicated for the second-line treatment of AIDS-related Kaposi's sarcoma, which is a cancer caused by infection with Kaposi sarcoma-associated herpesvirus. With conventional drug pricing systems, the price per unit of the drug is the same regardless of factors such as what type of cancer the patient is being treated for and how well the patient responds to the treatment. Likewise, drugs are priced independently of what other drugs or treatments are also prescribed as part of the patient's treatment regimen. For example, the price of a drug such as TAXOL® is the same, whether it is used alone or in combination with other drugs. Thus, a need exists for methods and systems for tailored or customizable drug pricing.

### SUMMARY

The disclosure according to some exemplary embodiments relates generally to an infrastructure for using electronic records of a patient's medical information (e.g., EMRs, EHRs, and/or PHRs). In some embodiments, the infrastructure may be used to determine drug and other treatment prices that can be different from listed prices based on various factors such as approved drug indication, whether and what other drug or drugs are used as part of the treatment regimen, a diagnosis and treatment plan, patients' responses to treatment, the organization and/or individual responsible for payment, among other factors. Advantageously, in some embodiments, systems and methods using the infrastructure can allow patients to receive drug and treatment prices based on the circumstances under which they received the treatment and can increase patients' access to drugs and other treatments through customized pricing that is more closely aligned to the value of their treatment. In addition, in some embodiments, the customized prices are lower than listed drug prices per unit. In conventional systems, a uniform price per unit is negotiated with payers resulting in uniform pricing regardless of drug uses and indications. In some embodiments of the disclosure, pricing varies based on multiple factors including the identity of the payer, the drug indications, concomitant treatments, and clinical results for a specific patient or patient population, amongst other factors.

According to some aspects, an electronic data infrastructure comprises a memory storing instructions; and a hardware processor to execute the instructions. The hardware processor executes instructions to identify a first electronic data record for a first patient based on a first identifier associated with the first electronic data record in a medical information electronic data repository. The hardware processor further executes instructions to analyze the first electronic data record to identify first data corresponding to patient information for the first patient based on a second identifier. The hardware processor also executes instructions to analyze the first electronic data record to identify second data corresponding to a first therapeutic intervention for the first patient based on a third identifier. Additionally, the hardware processor executes instructions to set a value for the first therapeutic intervention based at least in part on the first data and the second data and associate the value for the first therapeutic intervention with the first electronic data record.

In some embodiments, the first data comprises an approved indication treated with the first therapeutic intervention and the hardware processor further executes the instructions to determine the value for the first therapeutic intervention based at least in part on the approved indication treated with the first therapeutic intervention. In some embodiments, the hardware processor executes the instructions to determine the value by associating the second identifier and the third identifier with a corresponding second identifier and a corresponding third identifier associated with an agreed value table.

In some embodiments, the hardware processor executes the instructions to analyze the first electronic data record to identify third data corresponding to a second therapeutic intervention for the first patient based on a fourth identifier, the second therapeutic intervention is administered to the patient in combination with the first therapeutic intervention. The hardware processor also executes the instructions to determine the value for the first therapeutic intervention based at least in part on the first therapeutic intervention being administered in combination with the second therapeutic intervention.

In some embodiments, the first data comprises treatment results associated with administering the first therapeutic intervention and the hardware processor executes the instructions to determine the value for the first therapeutic intervention based at least in part on the treatment results associated with administering the first therapeutic intervention. In some embodiments, the first data comprises a duration the first therapeutic intervention has been administered and the hardware processor executes the instructions to determine the value for the first therapeutic intervention based at least in part on the duration the first therapeutic intervention has been administered.

In some embodiments, the hardware processor executes the instructions to determine a first payer value for the first therapeutic intervention for a first payer based at least in part on the first data and the second data and to determine a second payer value for the first therapeutic intervention for a second payer based at least in part on the first data and the second data.

In some embodiments, the first electronic data record is at least one of an EMR, an EHR, or a PHR. Additionally, in some embodiments, the first electronic record may be that of a payer. In some embodiments, the hardware processor executes the instructions to provide an interface for multiple payer systems.

In some embodiments, the hardware processor executes the instructions to receive agreed pricing data from at least one of a seller and a payer, determine the value for the first based at least in part on the agreed pricing data, and send the value to at least one of the seller and the payer. In some embodiments, the hardware processor executes the instructions to determine the value and send the value to the at least one of the seller and the payer without sharing the first data with the seller or the payer.

In some embodiments, the first therapeutic intervention is a drug. In some embodiments, the first therapeutic intervention is a first drug and the second therapeutic intervention is a second drug. In some embodiments, the first therapeutic intervention is a drug and the second therapeutic intervention is a non-drug intervention. In some embodiments, the hardware processor executes the instructions to analyze the first electronic data record to identify one or more of fourth data corresponding to a third therapeutic intervention for the first patient based on a fifth identifier, fifth data corresponding to a fourth therapeutic intervention for the first patient based on a sixth identifier, and sixth data corresponding to a fifth therapeutic intervention for the first patient based on a seventh identifier, and one or more of the third, fourth, and fifth therapeutic interventions are administered to the patient in combination with the first and second therapeutic interventions. The hardware processor also executes the instructions to determine the value for the one or more of the third, fourth, and fifth therapeutic interventions based at least in part on the one or more of the third, fourth, and fifth therapeutic interventions being administered in combination with the first and second therapeutic interventions. In some embodiments, the hardware processor executes the instructions to determine the value for one or more additional therapeutic interventions administered to the patient in combination with the first, second, third, fourth, and fifth therapeutic interventions, and determines the value of the one or more additional therapeutic interventions based at least in part on the one or more additional therapeutic interventions being administered in combination with the first, second, third, fourth, and fifth therapeutic interventions.

These and other aspects and embodiments of the disclosure are illustrated and described below.

### BRIEF DESCRIPTION OF THE DRAWINGS

Exemplary embodiments are described with reference to the following figures, which are presented for the purpose of illustration only and are not intended to be limiting.

FIG. 1 shows a method for using an electronic record infrastructure for drug pricing and reimbursement according to an exemplary embodiment.

FIG. 2A shows a drug price generator for use with an electronic record infrastructure according to some exemplary embodiments. FIG. 2B shows a drug price generator algorithm using an electronic record infrastructure according to some exemplary embodiments.

FIG. 3A shows an exemplary drug price determination using an electronic record infrastructure according to some exemplary embodiments. FIG. 3B shows an electronic record infrastructure for exemplary drug price processing network according to some exemplary embodiments.

FIG. 4 shows a patient electronic record table for use with an electronic record infrastructure according to an exemplary embodiment.

FIG. 5A and FIG. 5B show value-based price (VBP) and revenue split tables for use with an electronic record infrastructure according to an exemplary embodiment.

FIG. 6 illustrates an example of an electronic record table showing patient IDs, diagnosis codes, drug scans/treatment information, dates of UPC scans, approved treatments, approved indication codes, value-based pricing (VBP) regimens, and VBP splits in accordance with some embodiments.

FIG. 7 shows an example of a table showing sources of patient specific information that can be brought into a patient's EHR, EMR, or other health record according to an exemplary embodiment.

FIG. 8 shows an example of two value-based pricing schemes for use with an electronic record infrastructure according to an exemplary embodiment.

FIG. 9 shows an example of a value-based pricing scheme for use with an electronic record infrastructure according to an exemplary embodiment.

FIG. 10 shows an example of a block diagram of a system that can be used to implement the infrastructure described herein in accordance with some embodiments.

FIG. 11 shows an example of a block diagram of hardware that can be used to implement computers, servers, and/or databases described herein and/or certain components of FIG. 11, in accordance with some embodiments.

### DETAILED DESCRIPTION

The disclosure according to some exemplary embodiments relates generally to an infrastructure for using electronic records of patient's medical information (e.g., EMRs, EHRs, PHRs, and/or payers' electronic records). The infrastructure can be used, for example, to determine a drug price different from a listed per unit drug price based on various factors. According to some exemplary embodiments, a patient comes into a healthcare provider's office (e.g., clinic/physician's office or hospital) and an electronic record (e.g., EMR, EHR, and/or PHR) is created with data such as the patient's name, date of birth, identification, among other data. In some embodiments, a patient can create his or her own PHR, for example, using software on a desktop computer, laptop computer, smart phone, tablet, or other device. The physician's examination notes, the tests run, and other medical information can be added to the electronic record. Information about a diagnosis and treatment plan, as well as the patient's responses to treatment, can also be included. A pharmacist or physician can include drugs recommended for the patient's treatment, and labels such as Universal Product Code (UPC) bar code scans can be added to the patient's electronic record. As described in further detail below, software can collect relevant information and generate a recommended drug price by accessing information in the patient's electronic record and transforming it by applying one or more algorithms to determine appropriate customized, value-based price and/or discount applicable to the patient. One advantage of the systems and methods according to some exemplary embodiments is that they can be used without providing sellers access to private patient data. The systems and methods according to some exemplary embodiments allow patients to receive drug prices that can vary based on the underlying value provided, including the impact of the combination of two or more drugs by receiving a price tailored to the value of the treatment provided. Still a further advantage of the systems and methods according to some exemplary embodiments is that tailoring drug prices based on a particular patient's characteristics can increase access to drugs and other treatment. Advantageously, in some embodiments, multiple value-based prices can be negotiated between payers and manufacturers to better align pricing across patient types with the value provided by one or more drugs. In some embodiments, such techniques may advantageously more closely align prices with negotiated or agreed upon value for one or more drugs.

In some exemplary embodiments, software modules using technology (e.g., machine learning algorithms based on deductive reasoning) can run on software platforms at hospitals and clinics to create behind-the-scenes infrastructure to accurately match any treatment for any patient to its previously-agreed-upon value-based price and/or value-based scheme. In some exemplary embodiments, this may be accomplished with minimal or no incremental resources at the hospital, clinic, payer, or pharmaceutical company level, and without the need for the pharmaceutical company to receive patient level data. In some embodiments, the system and methods herein can accurately handle the speed, volume, and complexities of complex drug landscapes.

A problem with traditional drug pricing systems and methods is that the systems only provide for a single price per unit that is not varied based on factors such as the underlying value of the drug given the diagnosis, current standard of care, and other factors. Such systems lack the technical capability to take into account patients, diagnoses, concomitant therapies and other factors with factors such as dispensed drug volumes. For example, a drug's value can vary for different treatments. Additionally, some drugs are used in combination with other drugs and can have different values depending on what other drugs are used in the combination. Furthermore, different patients respond differently to different therapies, and different patients can be administered therapies for longer or shorter periods of time or for more or fewer courses of treatment. These and other factors can lead to mismatches between the value of the drug and the price charged (e.g., between the value of the drug for a particular approved indication and the price charged, between the value of the drug based on the other drugs administered in the combination and the price charged, and/or between the duration and/or results of the treatment and the price charged). According to some exemplary embodiments, the systems and methods disclosed herein provide the technical infrastructure that can be used, for example, for value-based pricing determination where different prices can be charged based on factors such as indication treated, whether and what other drugs are used as part of the treatment, how a patient responds to the treatment, the duration of the treatment, the number of courses of treatment, among other factors. Another advantage according to some embodiments is that the systems and methods disclosed herein can be used to maintain separate pricing for different payers. Thus, such systems and methods can be used to maintain different prices for different payers for a given set of factors such as indication, treatment outcome, etc. This can be advantageous, for example, when determining prices for different types of payers such as government payers (which may have certain mandated prices) and commercial payers (which may have different commercially negotiated prices). In some embodiments, such systems and methods can be used to maintain the confidentiality of prices negotiated with multiple payers (e.g., insurance companies) for each seller (e.g., pharmaceutical companies), such as in situations where multiple products from different manufacturers are used in combination.

The methods and systems for value-based drug pricing disclosed herein can be used for various drugs indicated for various diseases or conditions. According to some exemplary embodiments, a drug, such as a drug used to treat cancer, including but not limited to an immune checkpoint inhibitor (such as drugs that are anti-PD-1 antibodies, anti-PD-L1 antibodies, and anti-CTLA-4 antibodies, amongst others), may be used to treat various conditions or diseases. In some cases, it may be desirable to combine, for example, the checkpoint inhibitor drug with one or more other drugs. For example, in some cases, it may be desirable to combine two, three, four, five, six, or more drugs as part of a treatment regimen to treat various conditions or diseases, including but not limited to cancer. In some cases, these drugs may be supplied by different manufacturers. In further cases, multiple drugs may be provided by the same manufacturer.

For example, in some embodiments, the drugs for which the disclosed methods and systems can be used to determine value-based drug pricing include but are not limited to the following:

ATRIPLA® (efavirenz/emtricitabine/tenofovir disoproxil fumarate) (a combination of two nucleoside analog HIV-1 reverse transcriptase inhibitor (emtricitabine and tenofovir disoproxil fumarate) and one non-nucleoside HIV-1 reverse transcriptase inhibitor (efavirenz) indicated for treatment alone or in combination with other antiretroviral agents for the treatment of HIV-1 infection in adults and pediatric patients 12 years of age and older);

AZACTAM® (aztreonam injection)(a synthetic bactericidal antibiotic indicated for the treatment of various infections caused by susceptible Gram-negative microorganisms);

BARACLUDE® (entecavir)(a hepatitis B virus nucleoside analogue reverse transcriptase inhibitor indicated for the treatment of chronic hepatitis B virus infection in adults and pediatric patients two years of age and older with evidence of active viral replication and either evidence of persistent elevations in serum aminotransferase or histologically active disease);

COUMADIN® (warfarin sodium)(a vitamin K antagonist indicated for (1) prophylaxis and treatment of venous thrombosis and its extension, pulmonary embolism; (2) prophylaxis and treatment of thromboembolic complications associated with atrial fibrillation and/or cardiac valve replacement; and (3) reduction in the risk of death, recurrent myocardial infarction, and thromboembolic events such as stroke or systemic embolization after myocardial infarction);

DAKLINZA™ (daclatasvir)(a hepatitis C virus (HCV) NS5A inhibitor indicated for use with sofosbuvir, with or without ribavirin, for the treatment of chronic HCV genotype 1 or 3 infection);

DROXIA® (hydroxyurea)(an antimetabolite indicated to reduce the frequency of painful crises and to reduce the need for blood transfusions in patients with sickle cell anemia with recurrent moderate to severe painful crises);

ELIQUIS® (apixaban)(a factor Xa inhibitor indicated (1) to reduce the risk of stroke and systemic embolism in patients with nonvalvular atrial fibrillation; (2) for the prophylaxis of deep vein thrombosis (DVT), which may lead to pulmonary embolism (PE), in patients who have undergone hip or knee replacement surgery; and (3) for the treatment of DVT and PE and for the reduction in the risk of recurrent DVT and PE following initial therapy);

EMPLICITI™ (elotuzumab)(a Signaling Lymphocytic Activation Molecule Family member 7 protein (SLAMF7)-directed immunostimulatory antibody indicated in combination with lenalidomide and dexamethasone for the treatment of patients with multiple myeloma who have received one to three prior therapies);

ETOPOPHOS® (etoposide phosphate)(a topoisomerase inhibitor indicated for the treatment of patients (1) with refractory testicular tumors, in combination with other chemotherapeutic drugs; and (2) for the treatment of patients with small cell lung cancer, in combination with cisplatin, as first-line treatment);

EVOTAZ® (atazanavir and cobicistat)(a two-drug combination of atazanavir, a human immunodeficiency virus (HIV-1) protease inhibitor, and cobicistat, a CYP3A inhibitor, indicated for use in combination with other antiretroviral agents for the treatment of HIV-1 infection);

GLUCOPHAGE® (metformin hydrochloride) and GLUCOPHAGE® XR (metformin hydrochloride) EXTENDED RELEASE (oral anti-hyperglycemic drugs used as an adjunct to diet and exercise to improve glycemic control in adults and children with type 2 diabetes mellitus);

GLUCOVANCE® (glyburide and metformin hydrochloride)(two oral antio-hyperglycemic drugs indicated as an adjunct to diet and exercise to improve glycemic control in adults with type 2 diabetes mellitus);

HYDREA® (hydroxyurea)(an anti-metabolite indicated for the treatment of (1) resistant chronic myeloid leukemia; and (2) locally advanced squamous cell carcinomas of the head and neck, (excluding lip) in combination with concurrent chemoradiation);

KENALOG®-10 (triamcinolone acetonide)(a synthetic glucocorticoid corticosteroid with marked inflammatory action indicated (1) as adjunctive therapy for short-term administration (to tide the patient over an acute episode or exacerbation) in acute gouty arthritis, acute and subacute bursitis, acute nonspecific tenosynovitis, epicondylitis rheumatoid arthritis, synovitis, or osteoarthritis) for intra-articular of soft tissue administration; and (2) for alopecia areata; discoid lupus erythematosus; keloids; localized hypertrophic, infiltrated, inflammatory lesions of granuloma annulare, lichen planus, lichen simplex chronicus (neurodermatitis), and psoriatic plaques; necrobiosis lipoidica diabeticorum for intralesional administration. Kenalog®-10 Injection may also be useful in cystic tumors of an aponeurosis or tendon (ganglia).

KENALOG®-40 (triamcinolone acetonide)(a synthetic glucocorticoid corticosteroid with anti-inflammatory action indicated for intramuscular use for allergic states, dermatologic diseases, endocrine disorders, gastrointestinal disease, hematologic disorders, neoplastic diseases, nervous system conditions, ophthalmic diseases, renal diseases, and rheumatic disorders);

LYSODREN® (mitotane)(an adrenal cytotoxic agent indicated for the treatment of inoperable, functional or nonfunctional, adrenal cortical carcinoma);

MEGACE® (megestrol acetate)(a synthetic derivative of the naturally occurring steroid hormone, progesterone, indicated for the treatment of anorexia, cachexia, or an unexplained, significant weight loss in patients with a diagnosis of acquired immunodeficiency syndrome (AIDS));

NULOJIX® (belatacept)(a selective T-cell costimulation blocker indicated for prophylaxis of organ rejection in adult patients receiving a kidney transplant);

OPDIVO® (nivolumab)(a programmed death receptor-1 (PD-1) blocking antibody indicated for the treatment of numerous cancers, including unresectable or metastatic melanoma, adjuvant treatment of melanoma, metastatic non-small cell lung cancer, advanced renal cell carcinoma, classical Hodgkin lymphoma, recurrent or metastatic squamous cell carcinoma of the head and neck, locally advanced or metastatic urothelial carcinoma, microsatellite instability-high or mismatch repair deficient metastatic colorectal cancer, and hepatocellular carcinoma);

ORENCIA® (abatacept)(a selective T cell costimulation modulator indicated for (1) moderately to severely active rheumatoid arthritis in adults, either as monotherapy or concomitantly with Disease-Modifying Antirheumatic Drugs (DMARDS) other than tumor necrosis factor (TNF) antagonists; (2) moderately to severely active polyarticular juvenile idiopathic arthritis in patients two years of age and older, either as monotherapy or concomitantly with methotrexate);

PLAVIX® (clopidogrel bisulfate)(a P2Y12 platelet inhibitor (1) indicated to reduce the rate of myocardial infarction (MI) and stroke in patients with non-ST-segment elevation ACS (unstable angina (UA)/non-ST-elevation myocardial infarction (NSTEMI)), including patients who are to be managed medically and those who are to be managed with coronary revascularization. Plavix should be administered in conjunction with aspirin; (2) indicated to reduce the rate of myocardial infarction and stroke in patients with acute ST-elevation myocardial infarction (STEMI) who are to be managed medically; and (3) in patients with established peripheral arterial disease or with a history of recent MI or recent stroke, Plavix® is indicated to reduce the rate of MI and stroke.

PRAVACHOL® (pravastatin sodium)(an hdroxymethylglutaryl-CoA synthase (HMG-CoA) reductase inhibitor (statin) indicated as an adjunctive therapy to diet to:
Reduce the risk of MI, revascularization, and cardiovascular mortality in hypercholesterolemic patients without clinically evident congenital heart disease (CHD).
Reduce the risk of total mortality by reducing coronary death, MI, revascularization, stroke/transient ischemic attack (TIA), and the progression of coronary atherosclerosis in patients with clinically evident CHD.
Reduce elevated total cholesterol (Total-C), low-density lipoprotein cholesterol (LDL-C), apolipoprotein B (ApoB), and triglyceride (TG) levels and to increase high-density lipoprotein cholesterol (HDLC) in patients with primary hypercholesterolemia and mixed dyslipidemia.
Reduce elevated serum TG levels in patients with hypertriglyceridemia.
Treat patients with primary dysbetalipoproteinemia who are not responding to diet.

REYATAZ® (atazanavir)(a protease inhibitor indicated for use in combination with other antiretroviral agents for the treatment of HIV-1 infection for patients three months and older weighing at least 5 kg.

SPRYCEL® (dasatinib)(a kinase inhibitor indicated for the treatment of adult patients:
newly diagnosed Philadelphia chromosome-positive (Ph+) chronic myeloid leukemia (CIVIL) in chronic phase.
chronic, accelerated, or myeloid or lymphoid blast phase Ph+ CML with resistance or intolerance to prior therapy including imatinib.
Philadelphia chromosome-positive acute lymphoblastic leukemia (Ph+ ALL) with resistance or intolerance to prior therapy).
   Sprycel® is also indicated for the treatment of pediatric patients with Ph+ CML in chronic phase.

SUSTIVA® (efavirenz)(a non-nucleoside reverse transcriptase inhibitor indicated in combination with other antiretroviral agents for the treatment of HIV-1 infection in adults and in pediatric patients at least three months old and weighing at least 3.5 kg).

VIDEX® (didanosine)(a nucleoside reverse transcriptase inhibitor for use in combination with other antiretroviral agents for the treatment of HIV-1 infection);

VIDEX® EC (didanosine) DELAYED RELEASE (a nucleoside reverse transcriptase inhibitor for use in combination with other antiretroviral agents for the treatment of human immunodeficiency virus (HIV)-1 infection);

YERVOY® (ipilimumab)(a human cytotoxic T-lymphocyte antigen 4 (CTLA-4)-blocking antibody indicated for:
Treatment of unresectable or metastatic melanoma in adults and pediatric patients (12 years and older).
Adjuvant treatment of patients with cutaneous melanoma with pathologic involvement of regional lymph nodes of more than 1 mm who have undergone complete resection, including total lymphadenectomy); and/or

ZERIT® (stavudine)(a nucleoside reverse transcriptase inhibitor for use in combination with other antiretroviral agents for the treatment of human immunodeficiency virus (HIV)-1 infection).

As another example, in some embodiments, the drugs for which the disclosed methods and systems can be used to determine value-based drug pricing include but are not limited to the following: ABRAXANE; AFINITOR; ALECENSA; ALIMTA; AMJEVITA; ARZERRA; AVASTIN; CALQUENCE; ELOXATIN; ERBITUX; GAZYVA/GAZYVARO; GLEEVEC/GLIVEC; HEMLIBRA; HERCEPTIN; ICLUSIG; IDHIFA; IMFINZI; IRESSA; ISTODAX; JAKAFI; KADCYLA; KEYTRUDA; KISQALI; KYMRIAH; LENTI-D; LENTIGLOBIN; LYNPARZA; MEKINIST; MVASI; OTEZLA; PERJETA; POMALYST; REVLIMID; TAFINLAR; TAGRISSO; TARCEVA; TECENTRIQ; THALOMID; VELCADE; VIDAZA; VOTRIENT; XELODA; and ZYKADIA.

The pricing systems and methods disclosed herein may be used with any drug or drug combinations for any disease or condition. In some cases, the pricing systems and methods disclosed herein may be applied to other therapeutic interventions, such as but not limited to medical devices or cell-based therapies. As such, if the price of the drugs in the treatment combination are set in isolation from each other, the overall price of the treatment may be unintended or impractical. According to some exemplary embodiments, the systems and methods disclosed herein provide the technical infrastructure to set drug prices for a treatment involving one drug or multiple drugs, where the drug or drugs may be supplied from one company or from two or more companies. In some embodiments, prices for one or more therapeutic interventions for a patient determined through pricing systems and methods disclosed herein may be used with prices for one or more other therapeutic interventions.

According to some embodiments, the systems and methods disclosed herein may operate without the payer (e.g., an insurance company) or the seller (e.g., a pharmaceutical company) having access to confidential patient records to determine the value-based price.

According to some embodiments, the systems and methods disclosed herein may be used for tracking diagnosis and transactional data. In some embodiments, patient outcomes can also be tracked. Longitudinal data may be tracked, for example, by tracking a chain of events for a particular individual with a patient identifier.

According to some embodiments, after a patient meets with a healthcare provider, for example, at a clinic, hospital, or in a physician's office, an electronic record such as an EMR, EHR, and/or PHR is created. When the patient, for example, has further meetings or communicates with the healthcare provider and/or receives further treatment, the electronic record is updated. For example, the healthcare provider may use an electronic records system to update EMRs, EHRs, and/or PHRs. Additionally, patients may update their PHRs using computers, smartphones, tablets, and other devices. According to some embodiments, the price of the drug can be set based on patient outcomes. For example, as a patient receives treatments, the patient's electronic record (e.g., an EMR, EHR, PHR) can be updated with information about the outcome of the treatment. The systems and methods according to some embodiments can then determine the price for the drug based on the treatment outcome. In some embodiments, the price can be set according to a schedule of prices for different outcomes agreed upon between the payer (e.g., an insurance company, hospital, or government) or the seller (e.g., a pharmaceutical company). In some embodiments, if the patient stops treatment and/or switches to a different treatment or passes away, the price can be adjusted, a discount may be applied, and/or a refund may be issued.

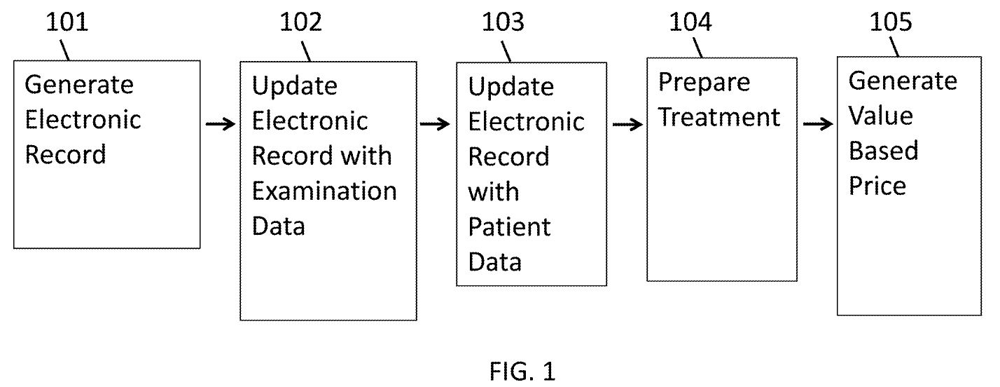

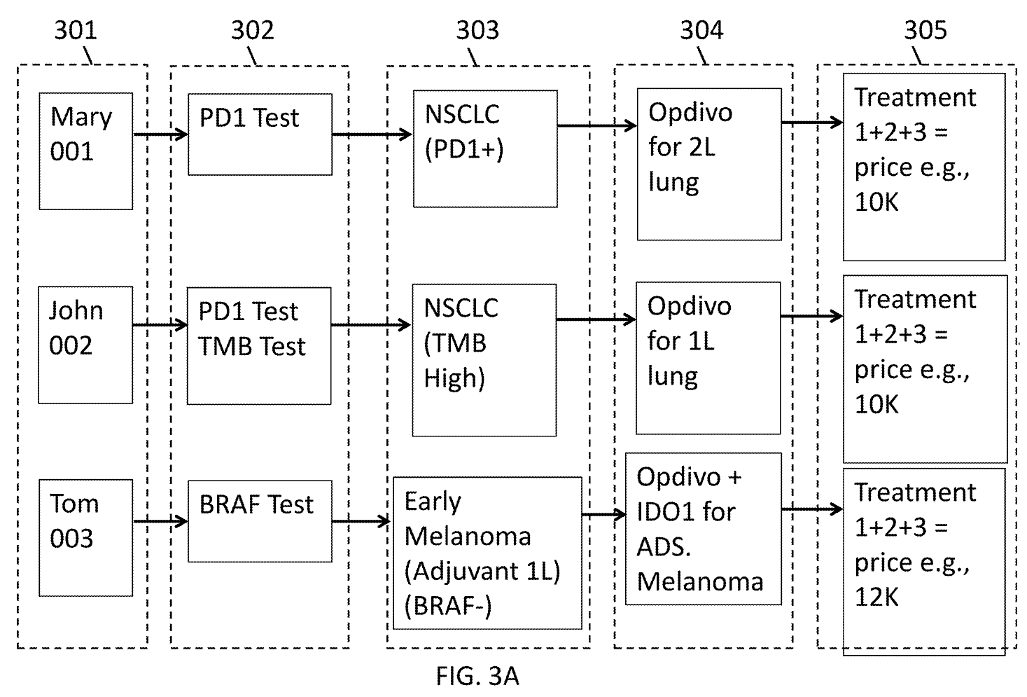

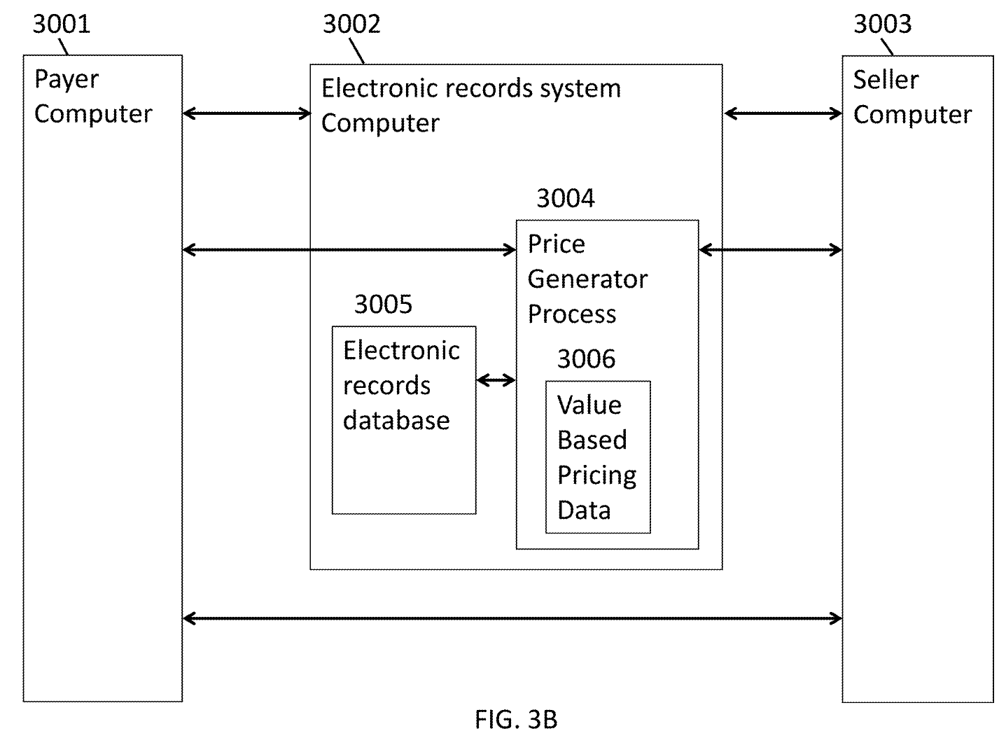

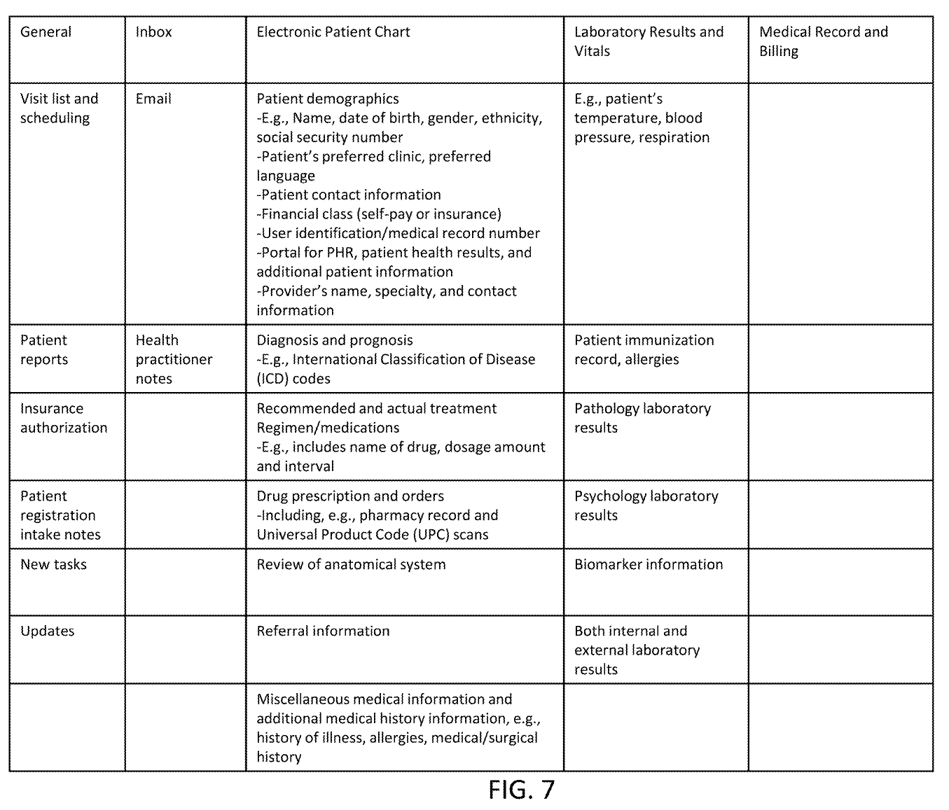

FIG. 1 shows use of the infrastructure for drug pricing and reimbursement according to an exemplary embodiment. At step 101 an electronic record such as an EMR, EHR, and/or PHR is generated and/or received. For example, when a patient visits a healthcare provider for the first time, an electronic record such as an EMR, EHR, and/or PHR can be generated for the patient. In some embodiments, electronic records can be created on computers, smartphones, or other devices, for example. The electronic record can include patient information such as a patient identifier. Electronic records can include medical records such as conditions, age, gender, health information such as blood pressure, pulse, other health factors such as smoking, drinking, status of biomarkers or other laboratory tests, other medications the patient is receiving, other health history information, for example. An EMR may, for example, contain a patient's medical history, diagnoses and treatments by a particular physician, nurse practitioner, specialist, dentist, surgeon or clinic. An EHR may, for example, store an electronic version of a patient's medical history that is maintained by a provider over time, and may include some or all of the administrative clinical data relevant to that person's care under a particular provider, including demographics, progress notes, problems, medications, vital signs, past medical history, immunizations, laboratory data and radiology reports. EHRs may be used to automate access to information and has the potential to streamline a clinician's workflow. EHRs may also support other care-related activities directly or indirectly through various interfaces, including evidence-based decision support, quality management, and outcomes reporting. Additionally, for example, a PHR may store a partial or complete summary of an individual's health and medical history based on data from many sources, including information entered by the individual (e.g., allergies, over the counter medications, family history, etc.). FIG. 7 shows an example of a table showing sources of patient specific information that can be brought into a patient's EHR, EMR, or other health record in accordance with some embodiments. In some embodiments, the infrastructure for drug pricing and reimbursement and/or a component thereof (e.g., price generator 3004 in FIG. 3B) can receive the generated electronic records.

Returning to FIG. 1, at step 102, the electronic record such as an EMR, EHR, and/or PHR can be updated with examination data such as physician notes, tests conducted, and test results, for example. In some embodiments, the infrastructure for drug pricing and reimbursement and/or a component thereof (e.g., price generator 3004 in FIG. 3B) can receive the updated electronic record with the examination data. At step 103, the electronic record can be updated with patient data such as diagnosis data and/or treatment data and/or a patient's biomarker data. In some embodiments, the diagnosis data may include one or more diagnoses. In some embodiments, the treatment data may include one or more treatment plans. In some embodiments, step 103 may comprise further processing an electronic record to combine data from some or all of the record and/or other sources such as other EMRs, EHRs, PHRs, and/or paper records, including data such as unstructured clinic notes and pathology reports, to provide an organized view of the patient's diagnosis, treatment, outcomes, and other medical information. In some embodiments, the infrastructure for drug pricing and reimbursement and/or a component thereof (e.g., price generator 3004 in FIG. 3B) can receive the updated electronic record with the patient data. At step 104, the patient treatment can be prepared, for example, by a physician providing a drug prescription and treatment regimen. In some embodiments, preparing the patient treatment can include combining one or more drugs according to the treatment plan. In some embodiments, a label such as a UPC code may be scanned to identify drugs prescribed for the patient and the patient's electronic record can be updated accordingly. In some embodiments, the infrastructure for drug pricing and reimbursement and/or a component thereof (e.g., price generator 3004) can receive a recommended treatment prepared for the patient. In some embodiments, at step 105, a value-based price can be determined for the drug based on data from the electronic record and/or preparation of the treatment. In some embodiments, step 105 may comprise performing a smart match to generate the price, as discussed further below.

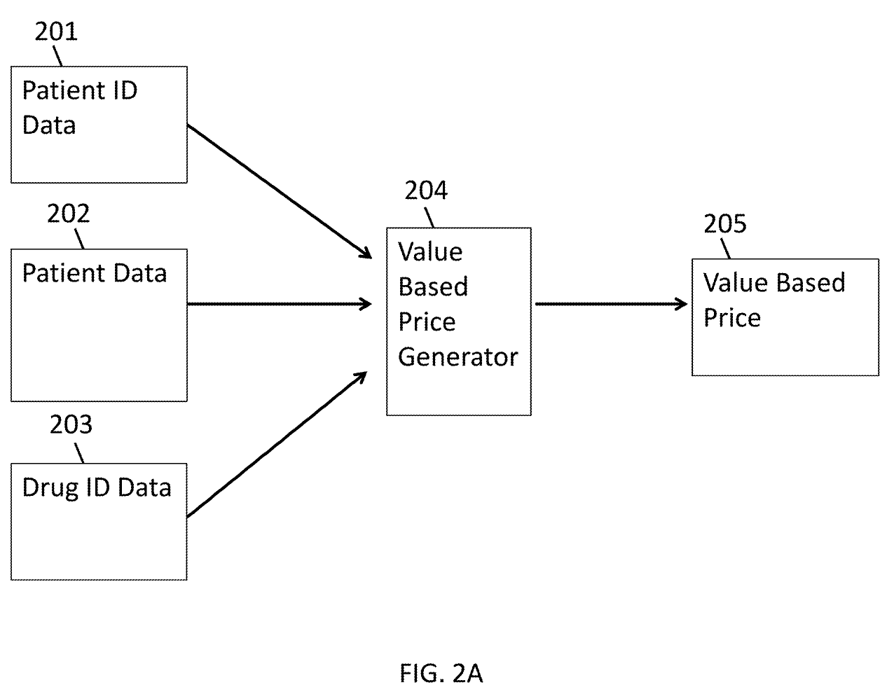

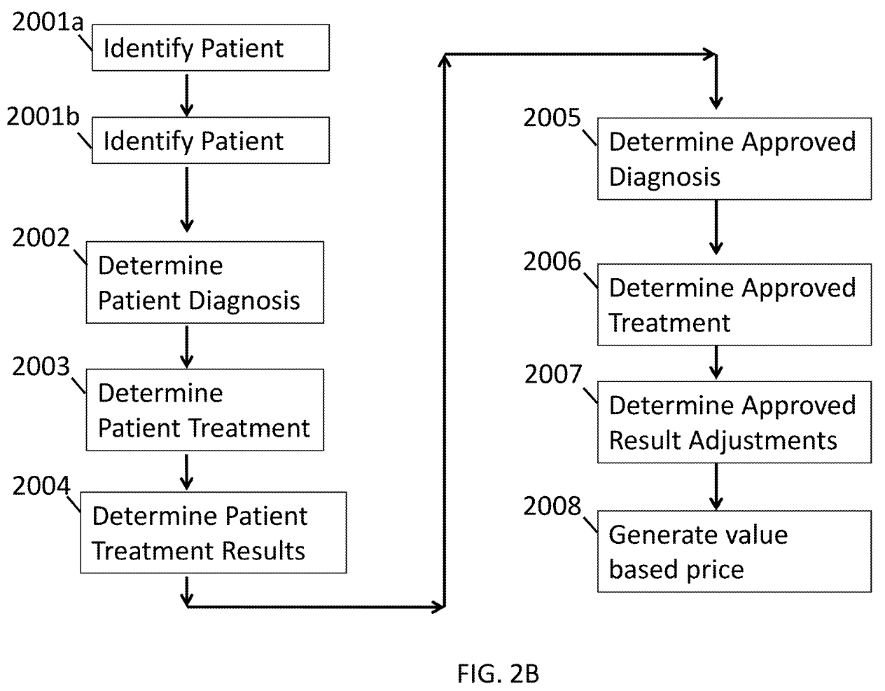

FIG. 2A shows a drug price generator 204 for use with an electronic record infrastructure according to some exemplary embodiments. The drug price generator 204 can receive data such as patient ID data 201, patient data 202 such as diagnosis and/or treatment data, and/or drug ID data 203. In some embodiments, the drug price generator 204 can obtain the patient ID 201 and the patient data 202 from the electronic record (e.g., EMR, EHR, PHR), associated with the patient. In some embodiments, the drug price generator 204 can obtain the drug ID data 203 from one or more labels scanned during treatment preparation. In some embodiments, the drug ID data 203 may also be stored with the electronic record and the drug price generator 204 may receive the drug ID along with other data stored in the electronic record. The drug price generator 204 can use the patient ID data 201, patient data 202 such as diagnosis and/or treatment data, and/or a drug ID data 203 to determine a value-based price scheme. For example, a payer (e.g., an insurance company) and a seller (e.g., a pharmaceutical company) may negotiate value-based price scheme that provides a value-based price that varies for a given drug based on factors including but not limited to what condition is being treated, what other drugs are being used as part of the treatment, and/or the results of the treatment. For example, the drug price generator 204 may determine the condition or conditions being treated from patient data 202 such as diagnosis and/or treatment data, what drug or drugs are being administered from the drug ID data 203, and what the results of the treatment are from the diagnosis and/or treatment data 202. The drug price generator 204 can then generate a value-based price for one or more patient IDs in the patient ID data according to an agreed price based on these factors. The drug price generator 204 can output the value-based price 205 associated with the patient ID data 201 to indicate the agreed price for the drug or drugs for the patient or patients associated with the patient ID data.

FIG. 2B shows a drug price generator algorithm for use with an electronic record infrastructure according to some exemplary embodiments. At step 2001a, a patient is identified, for example, based on a patient ID in the patient's electronic record (e.g., EMR, EHR, PHR). At step 2001b, the patient's payer is identified, for example, based on payer information in the patient's electronic record (e.g., EMR, EHR, PHR). At step 2002, a patient diagnosis is determined, for example, using an indication code in the patient's electronic record. At step 2003, patient treatment, such as drug prescriptions and surgical interventions are determined, for example, by processing a drug treatment identifier and/or other information stored in the patient's electronic record. In some embodiments, the drug treatment may be determined by processing a UPC code or other label scanned when preparing the drug treatment. At step 2004, patient treatment results are determined, for example, by reading a patient's electronic record (e.g., EMR, EHR, PHR) to determine the results of the treatment the patient receives. The patient's electronic record can be read in any suitable manner such as by requesting data from a database or by scanning a paper document and performing optical character recognition on the results of the scan. At step 2005, an approved diagnosis corresponding to the patient diagnosis is determined, for example, by processing data in a value-based pricing table to match an approved diagnosis in the value-based pricing table with the patient diagnosis. An approved diagnosis can be matched to a patient diagnosis in any suitable manner in some embodiments. For example, in some embodiments the most similar approved diagnosis in the table can be considered a match to the patient diagnosis. Diagnoses can be compared using medical codes, using natural language processing, using tables relating similar diagnoses, etc. in some embodiments. At step 2006, an approved treatment corresponding to the patient treatment is determined, for example, by processing data in a value-based pricing table to match an approved treatment in the value-based pricing table with a patient diagnosis. An approved treatment corresponding to a patient treatment can be determined in any suitable manner, for example, by identifying approved treatment(s) in the table that are linked to a matching approved diagnostic. At step 2007, an approved result adjustment is determined, for example, corresponding to the treatment results in the patient's electronic record. Approved result adjustment can be determined in any suitable manner in some embodiments. For example, in some embodiments, an approved result adjustment can be determined by accessing the patient's electronic record (e.g., EMR, EHR, PHR), matching it up to a specific value-based pricing agreement (from an electronic record storing value-based price agreements) between the manufacturer and the payer, and then applying the rules contained in that agreement to determine if an adjustment is warranted. At step 2008, a value-based price is determined by processing the approved diagnosis, approved treatment, and/or approved results adjustment.

FIG. 3A shows an exemplary use of the disclosed infrastructure for drug price determination according to some exemplary embodiments. In FIG. 3A, a computer system according to some embodiments (such as the system shown in FIG. 3B) uses Mary's electronic record (e.g., EMR, EHR, PHR) to identify her with patient ID 001, John's electronic record to identify him with patient ID 002, and Tom's electronic record to identify him with patient ID 003, as shown in 301. In 302, a programmed cell death protein 1 (PD1) test is administered for Mary, and the results including patient data, such as diagnosis and treatment data, are stored in her electronic record using a computer system according to some embodiments. Also in 302, a PD1 test and a tumor mutation burden (TMB) test are administered for John and the results are stored in his electronic record as patient data such as diagnosis and/or treatment data using a computer system according to some embodiments. Additionally, at 302 a BRAF gene mutation test is administered for Tom, and the results are stored in his electronic record as patient data such as diagnosis and/or treatment data using a computer system according to some embodiments. In this example, at 303, Mary receives a diagnosis of non-small cell lung cancer (NSCLC) based on a positive test result for the PD-1 protein test. John receives a diagnosis of NSCLC based on a high test result for the TMB test. Tom receives a diagnosis of early melanoma based on negative BRAF test results and treated with adjuvant therapy (surgery followed by chemotherapy or radiotherapy). At 304, Mary is prescribed Opdivo® (nivolumab) based on the PD-L2 biomarker associated with her lung cancer, John is prescribed Opdivo® (nivolumab) based on the PD-L1 biomarker associated with his lung cancer, and Tom is prescribed Opdivo® (nivolumab) and an indoleamine 2,3-dioxygenase (IDO1) inhibitor drug for his Adenocarcinoma (ADS) Melanoma.

At 305, in this example, a drug price generator such as the value-based generator 204 (shown in FIG. 2A) generates a drug price for each patient based on the diagnosis and the prescribed drugs. For example, based on the indications treated for Mary, the agreed price in this example is $10,000 for a three-drug treatment shown in the figure as “Treatment 1+2+3.” In this example, drug 1 is Opdivo® (nivolumab), and drugs 2 and 3 are two other agents used in combination with Opdivo® (nivolumab) to treat Mary's lung cancer. The seller (e.g., a pharmaceutical company) and the payer (e.g., an insurance company) agree on a combined price of $10,000 for the use of these three drugs together to treat this indication. The seller (e.g., a pharmaceutical company) and the payer (e.g., an insurance company) can also agree on an allocation of the total payment of $10,000 between the three drugs, for example, $4,000 for drug 1, $2,000 for drug 2, and $4,000 for drug 3. In some cases, the drugs may be supplied by multiple sellers, in which case an agreement can be made between each seller and the payer as to the drug prices, on a bilateral and/or multilateral basis. The agreed price can be based, for example, on what other drugs are used in the combination and what indication is being treated. The drug price generator of the computer system can, for example, process to determine that the agreed price for the three drugs is $10,000 by accessing Mary's electronic record using Mary's ID 001, process to determine which drugs have been prescribed and for what purpose, and calculate the agreed price for this combination of drugs and this treatment, for example, by accessing a database record with the agreed price or by performing a computation according to an agreed price determination formula. The drug price generator of the computer system according to some exemplary embodiments can then notify the seller (e.g., a pharmaceutical company) and the payer (e.g., an insurance company) of the drugs that are being used for treatment and the agreed price for the drugs for this particular treatment. Advantageously, the drug price generator of the computer system according to some exemplary embodiments can determine the agreed drug prices without sharing confidential medical information about the patient with either the seller (e.g., a pharmaceutical company) or the payer (e.g., an insurance company). In some embodiments, the drug price generator can be incorporated into an electronic records system (for example as software that runs on the medical records system) to improve the operation of the system by enabling it to determine a value-based drug price for treatments of particular conditions with particular combinations of drugs.

In some embodiments, the computer system can be used to determine refunds for a payer (e.g., an insurance company). Continuing with the previous example where the seller (e.g., a pharmaceutical company) and the payer (e.g., an insurance company) agreed on an allocation of the total payment of $10,000 between the three drugs, for example, $4,000 for drug 1, $2,000 for drug 2, and $4,000 for drug 3, there may be a difference between the wholesale costs of the drugs and the price generated by the system. For example, the wholesale price of drugs 1, 2, and 3 can be $5,000 each, for example. The payer (e.g., an insurance company) may enter into a rebate contract with the seller where the difference between the calculated price and the wholesale price is paid as a rebate. When the drugs are prescribed to Mary at the agreed price of $4,000 for drug 1, $2,000 for drug 2, and $4,000 for drug 3 for this particular treatment, the computer system can determine that the payer should receive a refund of $1,000 for drug 1, $3,000 for drug 2, and $1,000 for drug 3 based on the difference between the value-based price determined by the system and the wholesale price.

Continuing with 305 in FIG. 3A, a drug price generator such as the value-based price generator 204 (shown in FIG. 2A) generates a drug price for John. For example, based on the indications treated for John, the agreed price in this example is $10,000 for a three drug treatment shown in the figure as “1+2+3.” In this example, drug 1 is Opdivo® (nivolumab) and drugs 2 and 3 are two other agents used in combination with Opdivo® (nivolumab) to treat John's lung cancer. The seller (e.g., a pharmaceutical company) and the payer (e.g., an insurance company) agree on a combined price of $10,000 for the use of these three drugs together to treat this indication. The seller (e.g., a pharmaceutical company) and the payer (e.g., an insurance company) can also agree on an allocation of the total payment of $10,000 between the three drugs, for example, $3,000 for drug 1, $4,000 for drug 2, and $3,000 for drug 3 for this treatment case. In some cases, the drugs may be supplied by multiple sellers, in which case an agreement can be made between each seller and the payer as to the drug prices, on a bilateral and/or multilateral basis. The agreed price can be based, for example, on what other drugs are used in the combination and what indication are being treated. The drug price generator of the computer system can, for example, determine that the agreed price for the three drugs is $10,000 by accessing John's electronic record using John's ID 002, determining which drugs have been prescribed and for what purpose, and determining the agreed price for this combination of drugs and this treatment, for example, by accessing a database record with the agreed price or by performing a computation according to an agreed price determination formula. The drug price generator of the computer system according to some exemplary embodiments can then notify the seller (e.g., a pharmaceutical company) and the payer (e.g., an insurance company) of the drugs that are being used for treatment and the agreed price for the drugs for this particular treatment. Advantageously, the drug price generator of the computer system according to some exemplary embodiments can determine the agreed drug prices without sharing confidential medical information about the patient (e.g., John) with either the seller (e.g., a pharmaceutical company) or the payer (e.g., an insurance company).

As further shown in 305 in FIG. 3A, based on the indications treated for Tom, the agreed price in this example is $12,000 for a particular combination of three drugs, such as Opdivo® (nivolumab), an IDO1 inhibitor, and a third agent. The seller (e.g., a pharmaceutical company) and the payer (e.g., an insurance company) agree on a combined price of $12,000 for the use of these three drugs together to treat this indication. As illustrated in FIG. 3A, the price generator allows the seller and the payer to agree to a different total price for Tom's treatment of early melanoma as compared with John and Mary's treatments of NSCLC, even if the same three drugs were administered in each of these cases. In this example, the seller (e.g., a pharmaceutical company) and the payer (e.g., an insurance company) can also agree on an allocation of the total payment of $12,000 between the three drugs, for example, $4,000 for drug 1, $5,000 for drug 2, and $3,000 for drug 3 for this treatment case. In some cases, the drugs may be supplied by multiple sellers, in which case an agreement can be made between each seller and the payer as to the drug prices, on a bilateral and/or multilateral basis. The agreed price can be based, for example, on what other drugs are used in the combination and what indication are being treated. The drug price generator of the computer system can, for example, determine that the agreed price for the three drugs is $12,000 by accessing Tom's electronic record using Tom's ID 003, determining which drugs have been prescribed and for what purpose, and determining the agreed price for this combination of drugs and this treatment, for example, by accessing a database record with the agreed price or by performing a computation according to an agreed price determination formula. The drug price generator of the computer system according to some exemplary embodiments can then notify the seller (e.g., a pharmaceutical company) and the payer (e.g., an insurance company) of the drugs that are being used for treatment and the agreed price for the drugs for this particular treatment. Advantageously, the drug price generator of the computer system according to some exemplary embodiments can determine the agreed drug prices without sharing confidential medical information about the patient (e.g., Tom) with either the seller (e.g., a pharmaceutical company) or the payer (e.g., an insurance company). The drug price generator can thus provide the drug prices for each patient to the payer (e.g., an insurance company) and a seller (e.g., a pharmaceutical company). As illustrated in this example, the drug price can be determined for each patient without the payer (e.g., an insurance company) and the seller (e.g., a pharmaceutical company) accessing confidential patient data such as the diagnosis and treatment.

FIG. 3B shows an exemplary use of the disclosed infrastructure in a drug price processing network according to some exemplary embodiments. The drug price processing network comprises a payer 3001 such as an insurance company's computer system, an electronic records system 3002, and a seller 3003 such as a pharmaceutical company's computer system. The drug price processing network also comprises a price generator 3004. In some embodiments, the price generator 3004 is a software module that runs on the electronic records system 3002 or another computer system. The price generator 3004 may be incorporated into the electronic records system 3002 as depicted in FIG. 3B or may be separate. The electronic records system 3002 comprises electronic records 3005 and the price generator 3004 comprises valued based pricing 3006. Although the electronic records 3005 and the value-based pricing 3006 are depicted as stored in the electronic records system 3002 and the price generator 3004, respectively, one or both may also be stored separately. In some embodiments, the electronic records 3005 and/or the value-based pricing 3006 are stored in a database on the electronic records system 3002. In some embodiments, the electronic records 3005 and/or the value-based pricing 3006 are stored in a server and/or other cloud computing system.

According to some exemplary embodiments, the payer 3001 and the seller 3003 agree on value-based pricing 3006 for different treatments as described herein. The value-based pricing 3006 comprises agreed pricing based on a variety of factors such as treatment regimen, drug combination, indication treated, results of treatment (e.g., for performance-based pricing), among others. Patient medical information such as a patient ID, patient data such as diagnosis and treatment data, and drug ID information is stored in electronic records 3005. The price generators 3004 generates a value-based price for a particular patient by determining, for example, the approved diagnosis, approved treatment, and/or approved results corresponding to the patient diagnosis, patient treatment, and patient treatment results stored in the patient's electronic record and generating the value-based price corresponding to the approved diagnosis, approved treatment, and/or approved results. The price generator 3004 outputs the value-based price to the payer 3001 and the seller 3003. As illustrated in this example, the value-based price generator 3004 can generate a value-based price based on information in the electronic record without sharing confidential patient information with the payer 3001 or the seller 3003. The value-based price generator 3004 can also keep value-based pricing 3006 confidential from other payers 3001 and sellers 3003 that are not parties to particular value-based pricing agreements.

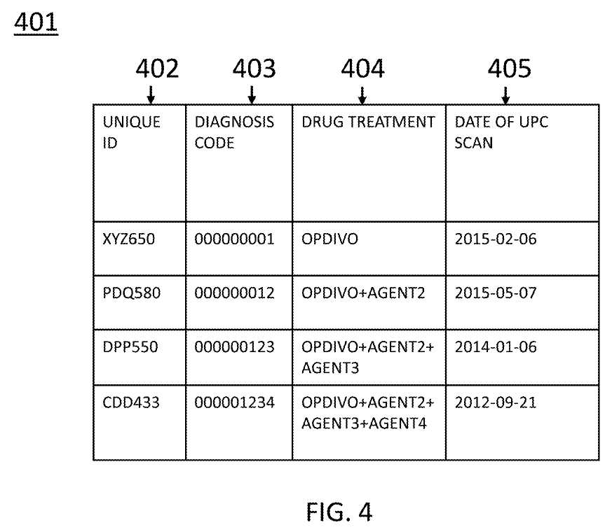

FIG. 4 shows a patient electronic record table 401 (e.g., from an EMR, EHR, or PHR) for use with an electronic record infrastructure according to an exemplary embodiment. The table can include data such as unique patient ID 402, diagnosis code 403, treatment information 404 (based, for example, on drugs scanned by the pharmacist) and the date 405 of the UPC scan. As discussed further below, a drug price generator according to some exemplary embodiments can access the data stored in the electronic record table 401 to determine value-based drug prices for particular treatments.

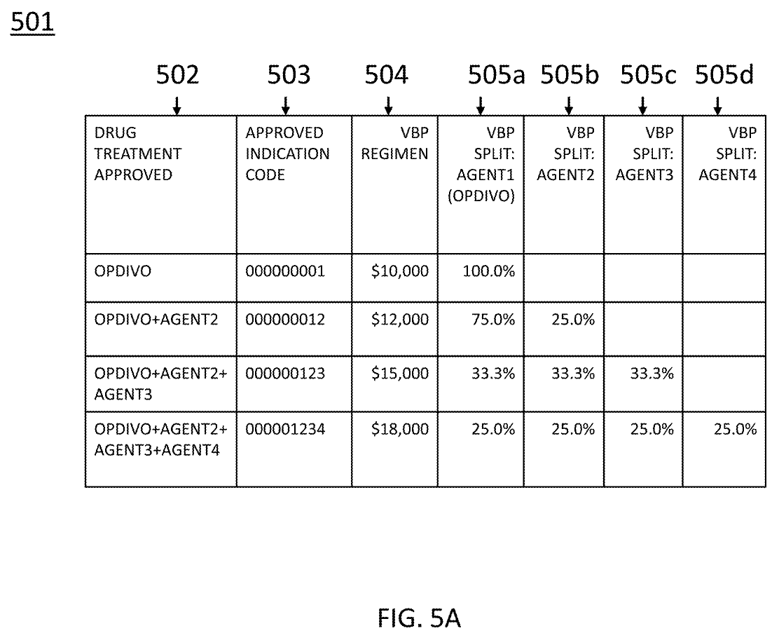

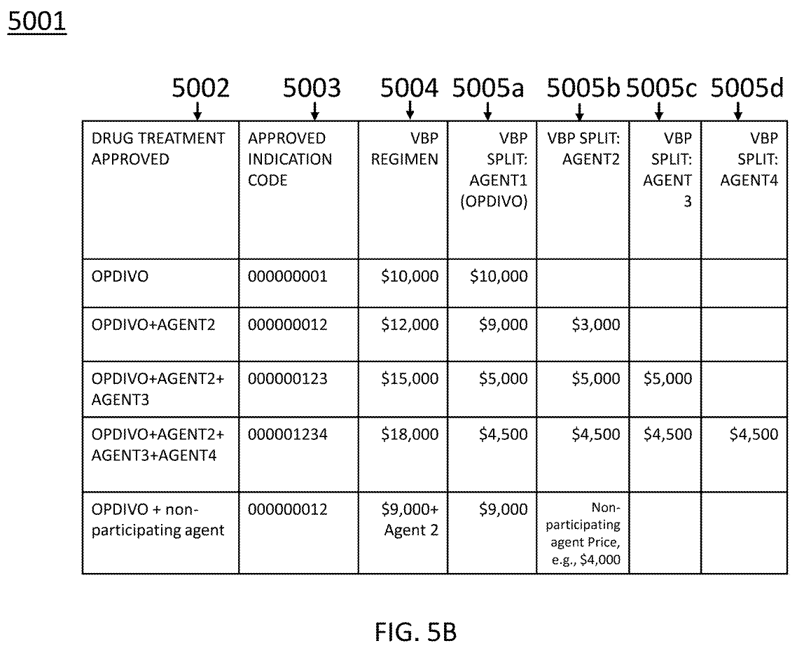

FIG. 5A shows a value-based price (VBP) and revenue split table 501 for use with an electronic record infrastructure according to an exemplary embodiment. In some embodiments, the table 501 may be included in an electronic record. In some embodiments, the table 501 may be stored separately from an electronic record. And in still further embodiments, portions of the data in table 501 may be stored in an electronic record and portions may be stored separately from an electronic record. Table 501 has a list of drug combinations and the indications for which they are approved, alongside current net prices for the treatment and proposed revenue splits by agent in the treatment. Column 502 identifies the drug combinations. Column 502 may also store a UPC code associated with each drug. Column 503 provides a code for the approved indication for the drug combination in column 502. For example, approved indication code 000000001 can correspond to NSCLC. The table also includes a value-based price (VBP) 504 agreed by the manufacturer(s) of the drug(s) and the insurance company for the drug regimen. Columns 505a-505d provide, for this example, the percentage of the agreed price (from column 504) associated with each of the drugs in the regimen. For example, in row 1, the drug for treatment of indication 000000001 is Agent 1 (Opdivo® (nivolumab)) and the supplier of this agent receives 100% of the agreed price of $10,000 shown in column 504. In row 2, the supplier of Agent 1 receives an agreed price of 75% of $12,000 when used in combination with Agent 2 for approved indication 000000012 and the supplier of Agent 2 receives an agreed price of 25% of $12,000 for this indication and treatment combination. Row 3 shows a combination therapy of three drugs, where a total price of $15,000 is divided three ways between the suppliers of agents 1, 2, and 3, with each receiving an agreed share of 33% of the agreed total price for use of each drug in this combination to treat indication 000000123. Row 4 shows a combination therapy of four drugs, where a total price of $18,000 is divided four ways between the suppliers of agents 1, 2, 3, and 4, with each receiving an agreed share of 25% of the agreed total price for use of each drug in this combination to treat indication 000001234. As illustrated in these examples, the total price can be divided evenly or unevenly between the various suppliers. In some embodiments, the price generator receives the UPC(s) of the drug treatment approved 502 and the approved indication code 503 from the table 501. In some embodiments, electronic records may contain standardized codes such as those published by Health Level Seven and other organizations. In some embodiments, any suitable data processing techniques can be used to convert data from one format to another and/or to provide interoperability between systems using different electronic record standards and/or non-standardized records. The price generator determines the VBP 504 and the split, based on columns 505a-505d. The price generator then generates the price of the drug due to each supplier (e.g., Agent1, Agent2, Agent3, and Agent4). In this manner, the system can determine the value-based price for the patient, with respect to the payer (e.g., an insurance company) and the seller(s) (e.g., a pharmaceutical company) without sharing patient confidential information.

FIG. 5B shows a further example of a value-based price (VBP) and revenue split table 5001 for use with an electronic record infrastructure according to an exemplary embodiment. In some embodiments, the Table 5001 may, for example, show actual values as illustrated in FIG. 5B rather than or in addition to percentages, as shown in FIG. 5A. For example, in FIG. 5B, columns 5005a-5005d provide, for this example, the portion of the agreed price (from column 5004) associated with each of the drugs in the regimen. For example, in row 1, the drug for treatment of indication 000000001 is Agent 1 (Opdivo® (nivolumab)) and the supplier of this agent receives 100% ($10,000) of the agreed price of $10,000 shown in column 5004. In row 2, the supplier of Agent 1 receives an agreed price of $9,000 when used in combination with Agent 2 for approved indication 000000012 and the supplier of Agent 2 receives an agreed price of $3,000 for this indication and treatment combination. Row 3 shows a combination therapy with three drugs, where a total price of $15,000 is divided three ways between the suppliers of agents 1, 2, and 3, with each receiving an agreed price of $5,000 for use of each drug in this combination to treat indication 000000123. Row 4 shows a combination therapy with four drugs, where a total price of $18,000 is divided four ways between the suppliers of agents 1, 2, 3, and 4, with each receiving an agreed share of $4,500 for use of each drug in this combination to treat indication 000001234. As illustrated in these examples, the total price can be divided evenly or unevenly between the various drug suppliers. In some embodiments, the price generator receives the UPC(s) of the drug treatment approved 5002 and the approved indication code 5003 from the table 5001. In some embodiments, electronic records may contain standardized codes such as those published by Health Level Seven and other organizations. In some embodiments, any suitable data processing techniques can be used to convert data from one format to another and/or to provide interoperability between systems using different electronic record standards and/or non-standardized records. The price generator determines the VBP 5004 and the agreed price shown in columns 5005a-5005d due to each supplier (e.g., Agent 1, Agent 2, Agent 3, and Agent 4). In this manner, the system can determine the value-based price for the patient, with respect to the payer (e.g., an insurance company) and the seller(s) (e.g., a pharmaceutical company) without sharing patient confidential information.

In some embodiments, the value-based price generator may generate a value-based price for a treatment used in combination with a non-participating treatment. For example, in some cases, the supplier of Agent 2 may be a supplier that is not participating in the value-based pricing system. The supplier of Agent 2 may instead, for example, sell the drug for a list price or a separately negotiated price. As shown in FIG. 5B, in row 5, the supplier of Agent 1 receives an agreed price of $9,000 when used in combination with a non-participating Agent 2, as shown in column 5004. The supplier of Agent 2 receives the separately determined or negotiated price, such as a list price of $4,000. Columns 5005a and 5005b in this example show the split between Agents 1 and 2, with Agent 1 receiving the value-based price of $9,000 and Agent 2 receiving a separately determined price, such as $4,000.

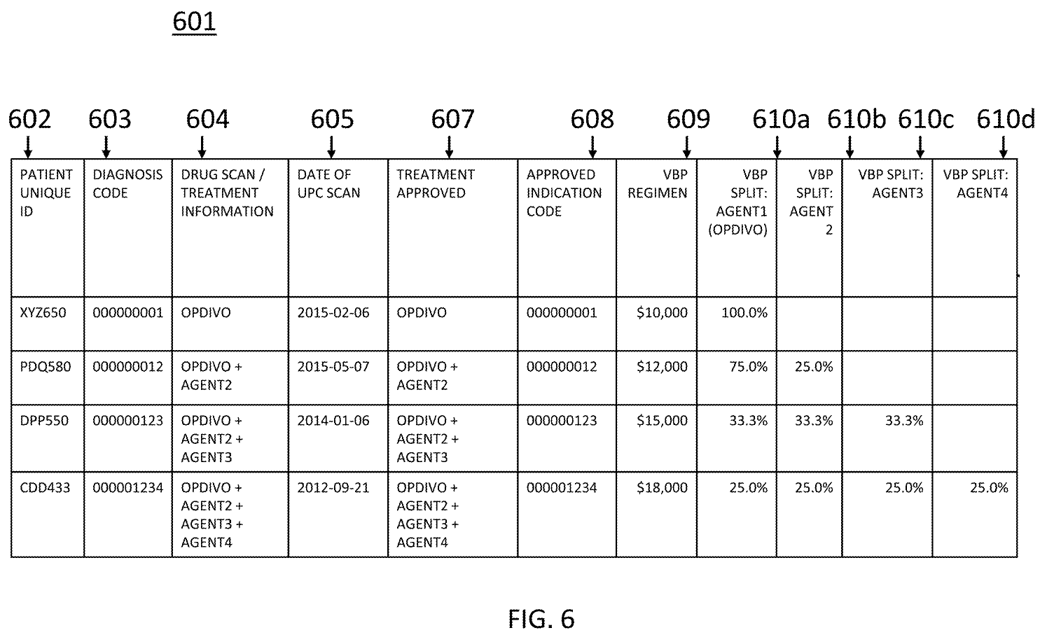

FIG. 6 illustrates an example of an electronic record table showing patient IDs, diagnosis codes, drug scans/treatment information, dates of UPC scans, approved treatments, approved indication codes, value-based pricing (VBP) regimens, and VBP splits in accordance with some embodiments. In a computer system according to some embodiments, a price generator can match patients to value-based price schemes. For example, in some embodiments, a patient unique ID 602, a diagnosis code 603, and a drug scan 604 can be used to determine a value-based price that corresponds to the user based on the patient unique ID indicating that the patient qualifies for the value-based price (e.g., because the patient is insured by a payer corresponding to the value-based price), based on the value-based price having an indication corresponding to the diagnosis code, and/or based upon the drug scan corresponding to a drug corresponding to the value-based price. In some embodiments, the price generator receives unique patient IDs 602, diagnosis code 603, treatment information 604 (based, for example, on drugs scanned by the pharmacist) and the date 605 of the UPC scan from the patient electronic record table 601. The price generator can determine one or more value-based prices by matching the diagnosis code 603 to the approved indication code 608 and/or by matching the UPC scan 604 to the UPC of drug treatment approved 607. In some embodiments, patients with particular conditions can be matched to agreed drug prices for treatment of those conditions with particular drug combinations. This matching can be performed in any suitable manner. For example, in some embodiments, patient information such as diagnosis and/or other medical factors, treatments or treatment combinations, duration of therapy, or outcomes can be compared to conditional pricing information to identify or calculate the pricing that is applicable to the individual treatment. The price generator determines the value-based price for the regimen 609 and the agreed split between agents 610a-610d to determine the price due for each agent. The price generator also shares the value-based price due for each agent with the payer (e.g., an insurance company) and the sellers (e.g., the manufacturers of Agents 1-4). In some embodiments, different patient electronic record tables 601 may be used with one or more different payers.

In some embodiments, the system can be used to provide for the full cycle of disease treatment for the patient. At each stage in the patient's treatment, there can be a specific approved FDA indication in some embodiments. In some embodiments, pricing for treatment without a specified value-based price may be determined using other pricing techniques. In some embodiments, value-based prices may be set in combination with one or more other value-based prices and/or in combination with one or more prices determined according to other pricing techniques. In some embodiments, the price generator can match each diagnosis code to FDA approved indication. For example, cancer drugs can have different FDA approved indications for each stage where they are used for treatment. The price generator of the computer system according to some embodiments can provide different value-based prices at each stage of treatment based on unique indication codes associated with those stages. For example, a payer and a seller can agree to a price of $10,000 for treatment during a first stage of treatment, $15,000 for treatment during a second stage of treatment, and $12,000 for treatment during a third stage of treatment. The price generator determines the value-based price for the drug during the first stage of treatment by identifying the approved FDA indication corresponding to use of the drug for treatment during the first stage in the patient's electronic record. The price generator then generates a price of $10,000 based on the agreed price for treatment during the first stage. The price generator also notifies the payer and seller that the agreed price for the drug treatment is $10,000. This allows the price generator to provide the agreed price for a particular treatment to the payer and seller without the need to provide the payer and seller with access to the patient's confidential medical information. Then, during the second stage of treatment, the price generator detects the FDA approved indication based on the code associated with the second stage of treatment in the patient's electronic record and determines the price for this treatment is $15,000 based on the agreed price for stage 2 treatment. The price generator can then inform the payer and seller of the agreed price without sharing confidential patient information. In the third stage of treatment, the price generator identifies the FDA approved indication based on the code for use of the drug for stage three treatment and determines the corresponding value-based price agreed to by the seller and the payer for stage 3 treatment. The price generator then informs the seller and the payer of the value-based price. In some embodiments, the price generator may inform the seller and the payer of the value-based price without sharing the value-based price with the physician, and in other embodiments, the value-based price may be shared with the physician.

In some embodiments, the system can be used to allow the same drug to have different prices for different diseases (e.g., different price for different types of cancer). The system can also be used to allow different prices when used with different drugs. For example, a payer and a seller could agree that drug 1 costs $10,000 when used to treat disease A and costs $15,000 when used to treat disease B. A payer and a seller could also agree that drug 1 costs $6,000 when used in combination with drug 2 to treat disease A and costs $8,000 when used in combination with drug 2 to treat disease B. The drug price generator can then determine the value-based price for a particular treatment for a particular patient by determining the agreed price for a particular drug or combination of drugs to treat a particular disease based, for example, on the diagnosis code and approved drug treatment in the patient's electronic record.

In some embodiments, price determinations can be made in real time using the disclosed infrastructure. For example, when a patient electronic record is updated with a diagnosis and a prescription for a particular drug or combination of drugs, the drug price generator can calculate the agreed value-based price based on the drug or combination of drugs and/or the indication treated. In some embodiments, the drug or combination of drugs can be identified based on a UPC or other code scanned, for example by a pharmacist, when preparing the drug treatment for the patient. In some embodiments, by determining prices in real time, patients can be provided the price of a combined treatment, without delay or waiting for rebates provided after a price is determined at a later date. In some embodiments, rebates can be provided to adjust prices based on factors such as the indication treated, the results of the treatment, and/or what combination of drugs were used for the treatment. For example, in some embodiments, the final price for a given treatment is a function of the underlying agreement with a particular payer. Differences between the final price and the acquisition cost of the individual product(s) used in the treatment can be refunded based on the agreement with the payer. This may, for example, include rebates paid in aggregate for a time period (e.g., quarterly), credits on future purchases, or other methods as defined in the manufacturer(s) agreement(s) with the payer.

In some embodiments, the price generator maintains the confidentiality of patient medical records, for example, by generating drug prices without sharing confidential patient data with the drug provider (e.g., a pharmaceutical company) or the payer (e.g., an insurance company). Additionally, the price generator maintains the confidentiality of pricing agreements between the payers and drug providers, for example, by generating drug prices for a payer and a drug provider without sharing the confidential pricing agreements between the payer and the drug provider with other payers and drug providers. For example, as illustrated above, a price generator according to an exemplary embodiment is incorporated into an electronic records system. The price generator accesses the electronic records for a patient corresponding to a patient ID and determines what indications are being treated and what drug or combination of drugs are being used for the treatment. In some embodiments, the drug price generator also stores agreed prices between payers and drug providers. The generator further stores agreed formulas for calculating agreed prices between payers and drug providers. The price generator uses the data in the electronic record (e.g., the condition treated and the drug or combination of drugs used in the treatment) to determine the value-based price based on the agreed price for such treatment. The price generator then provides the value-based price to the payer and the drug provider without sharing confidential patient information. Likewise, the price generator protects the confidentiality of the agreed prices between the payers and the drug providers. For example, the price generator determines the value-based priced based on the data in the electronic record without, for example, sharing the agreed prices with the hospital or with other payers.

In some embodiments, the price generator has different pricing agreements with different payers and/or with different categories of payers (e.g., Commercial program, Government program 1, Government program 2, other category of customer). For example, a seller has agreements to sell drug 1 for $8,000 to Veterans Affairs (VA) hospitals, for $12,000 to a first group of private hospitals, for $10,000 to a second group of private hospitals, for $9,000 for Medicare, and for $8,000 for Medicaid, among others. In some embodiments, the seller also has unique agreements with different buyers for different drug combinations and/or treatment of different conditions. When a patient is treated, the price generator generates the agreed valued based price in part based on the payer. The price generator also uses drug acquisition costs as an input to reconcile acquisition costs with the final agreed price with a payer for a particular patient's treatment, for example, by providing appropriate refunds and/or credits.

In some embodiments, the price generator provides automated reporting. For example, some payers such as federal supply payers have detailed reporting requirements. By providing automated reporting, the price generator reduces or relieves sellers and/or payers of the burden of reporting requirements for different payers and other entities. In some cases, advantageously, this reduces or eliminates data tracking for governmental and other reporting. In some embodiments, the full financial terms are supplied to individual manufacturers and/or payers, including but not limited to: acquisition costs, final reimbursed amount, class of customer, payer identification, among others, to enable automation of government reporting requirements. In some embodiments, the price generator reports aggregated rebates due to a payer.

In some embodiments, the price generator is used for other price determinations. For example, the price generator determines a value-based price for other treatments such as physical therapy and/or surgery. In some embodiments, the price generator determines a value-based price based on both drug treatments and non-drug treatments. For example, the price generator determines value-based prices for drugs and surgery in a combined treatment regimen.

In some embodiments, the disclosed infrastructure is used to facilitate price negotiation and agreements between payers and providers. This overcomes problems of negotiating prices independently with each provider, which can lead to a combined price for treatments involving multiple drugs that exceeds what the payer is willing to pay. For example, if a payer were willing to pay $15,000 for a treatment, and providers 1 and 2 offer drug treatments for $10,000 each, negotiating prices independently would make it difficult to arrive at an agreed price because the combined price of the drugs from each supplier is $20,000 and the payer is only willing to pay $15,000. In some embodiments, the disclosed systems and methods facilitates price negotiation and agreements by determining that the manufacturer of Drug 1 would be willing to supply one of the two drugs for $8,000 when used as part of the combined treatment and that the manufacturer of the Drug 2 would be willing to provide Drug 2 for $7,000 when used as part of the combined treatment. In some embodiments, the disclosed systems and methods can advantageously facilitate pricing and rebate calculations.

In some embodiments, multiple providers provide the same drug and the disclosed infrastructure can be used to facilitate price negotiation and agreements between payers and providers regarding classes of drugs available from multiple providers. For example, an exemplary HIV regimen includes two product classes, class 1 and class 2. Manufacturers of “class 1” can agree to a price of X dollars when used with “class 2.” Manufacturers of “class 2” can agree to a price of Y dollars when used with “class 1.” When the regimen is encountered in the system (e.g., a patient using “class 1” and “class 2” together), the system determines that the price is X plus Y dollars and that X dollars is the net price for class 1 (irrespective of manufacturer) and Y is the net price for class 2 (irrespective of manufacturer). In some embodiments, the disclosed systems and methods advantageously facilitate pricing and rebate calculations for classes of drugs.

In some embodiments, the disclosed infrastructure is used to provide automatic ordering and inventory management. For example, in some embodiments, the system is used to automate a request to a wholesaler or distributor that a particular product was used (e.g., 100 mg vial of Opdivo® (nivolumab)) and request a replacement stock be shipped.

The steps described herein according to one or more embodiments are exemplary. In some embodiments, steps disclosed herein may be performed in any order, unless otherwise indicated. In some embodiments, one or more steps disclosed herein may be omitted or repeated.

In some embodiments, the exemplary embodiments disclosed here (including, e.g., the price generator systems and methods) can be implemented in software stored in memory. The software can run on a hardware processor capable of executing computer instructions or computer code. In some embodiments, the hardware processor comprises a general-purpose hardware processor and/or hardware using an application specific integrated circuit (ASIC), programmable logic array (PLA), digital signal processor (DSP), field programmable gate array (FPGA), or any other integrated circuit. The hardware processor suitable for the execution of a computer program includes, by way of example, both general and special purpose microprocessors, digital signal processors, and any one or more hardware processors of any kind of digital computer. Generally, the hardware processor receives instructions and data from a read-only memory or a random-access memory or both.

In some embodiments, disclosed method steps (including, e.g., those for price generation) are performed by one or more hardware processors executing a computer program to perform functions of the exemplary embodiments by operating on input data and/or generating output data. In some embodiments, one or more of the components are also implemented in hardware. The systems and methods of according to some exemplary embodiments (including, e.g., the price generator systems and methods) are implemented in digital electronic circuitry, or in computer hardware, firmware, software, or in combinations of them.

In some embodiments, the systems and methods disclosed herein (including, e.g., the price generator systems and methods) are implemented in a computing device that is operatively coupled to external equipment, for example medical devices and patient diagnostic equipment, or to a communications network. Computer-readable storage devices suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks.

In some embodiments, the computing device (including, e.g., the price generator systems) is a computer, tablet, smart phone, or other mobile device. In some embodiments, the computing device (including, e.g., the price generator systems and methods) includes a server, for example, for remotely storing pricing information.

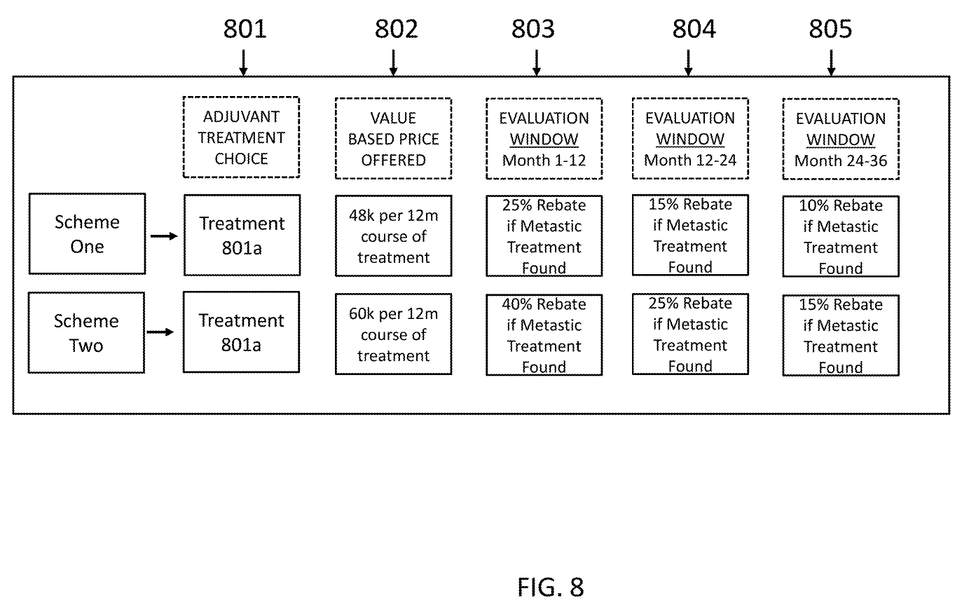

FIG. 8 shows an example of two value-based pricing schemes for use with an electronic record infrastructure according to an exemplary embodiment. In this example, a Patient receives treatment 801a (e.g., adjuvant therapy with Opdivo® (nivolumab)) after complete resection of stage III/IV melanoma. FIG. 8 shows two outcomes-logic-based-schemes to demonstrate Opdivo® (nivolumab)'s ability to delay recurrence. As shown in 801, the treatment choice for scheme one is treatment 801a (e.g., OPDIVO® (nivolumab)). The electronic record infrastructure according to an exemplary embodiment identifies the adjuvant treatment choice from the patient's electronic record. As shown at 802, the electronic record infrastructure determines a value-based price offered for scheme one. In this example, the value-based price for adjuvant is $48,000 per twelve-month course of treatment. The electronic record infrastructure calculates the value-based price based on factors such as the indication and stage of treatment (e.g., treatment after complete resection of stage III/IV melanoma). As shown at 803, in the scheme one example, the electronic record infrastructure uses an evaluation window, during months one through twelve of treatment in this example, to determine if a metastatic treatment was found on the electronic record for the patient during this window of time. The electronic record infrastructure then, for example, determines that a 25% rebate should be paid, on a patient level, if a metastatic treatment was found on the electronic record for the patient during this window of time. In this example, a metastatic treatment implies that a patient had recurrence of disease post adjuvant treatment with Opdivo® (nivolumab). Thus, the electronic record infrastructure adjusts the value-based price in this example via a rebate based on the recurrence of disease post adjuvant treatment with Opdivo® (nivolumab) during a first window.

As shown in 804, in the scheme one example, the electronic record infrastructure uses a second evaluation window, during months twelve through twenty-four of treatment in this example, to determine if a metastatic treatment was found on the electronic record for the patient during this second window of time. The electronic record infrastructure, for example, determines that a 15% rebate should be paid, on a patient level, if a metastatic treatment was found on the electronic record for the patient during this second window of time. Thus, the electronic record infrastructure furthers adjust the value-based price in this example via a rebate based on the recurrence of disease post adjuvant treatment with Opdivo® (nivolumab) during a second window.

As shown in 805, in the scheme one example, the electronic record infrastructure uses a third evaluation window, during months 24 through 36 of treatment in this example, to determine if a metastatic treatment was found on the electronic record for the patient during this third window of time. The electronic record infrastructure, for example, determines that a 10% rebate should be paid, on a patient level, if a metastatic treatment was found on the electronic record for the patient during this third window of time. Thus, the electronic record infrastructure further adjusts the value-based price in this example via a rebate based on the recurrence of disease post adjuvant treatment with Opdivo® (nivolumab) during a third window.

As further shown in FIG. 8, scheme two illustrates a further exemplary value-based pricing scheme for use with an electronic record infrastructure according to an exemplary embodiment. In the scheme two example, as shown at 801 and 802, the electronic record infrastructure determines that the value-based price for treatment 801a (e.g., the adjuvant treatment choice OPDIVO® (nivolumab)) is $60,000 per twelve-month course of treatment, rather than $48,000 in the scheme one example. As shown at 803, 804, and 805, the rebates if metastatic treatment is found are 40%, 25%, and 15% during the first, second, and third treatment windows respectively. Thus, schemes one and two in FIG. 8 illustrate two exemplary value-based pricing schemes, where scheme one has a higher initial price and lower performance-based rebate percentages and scheme two has a higher price and higher performance-based rebate percentages. Schemes one and two are exemplary. In other exemplary embodiments, the electronic record infrastructure determines different value-based prices, for example, by using different prices for different courses of treatment, by applying different agreed discounts, by applying different treatment windows, by applying different performance-based criteria, and/or by applying other factors discussed herein.

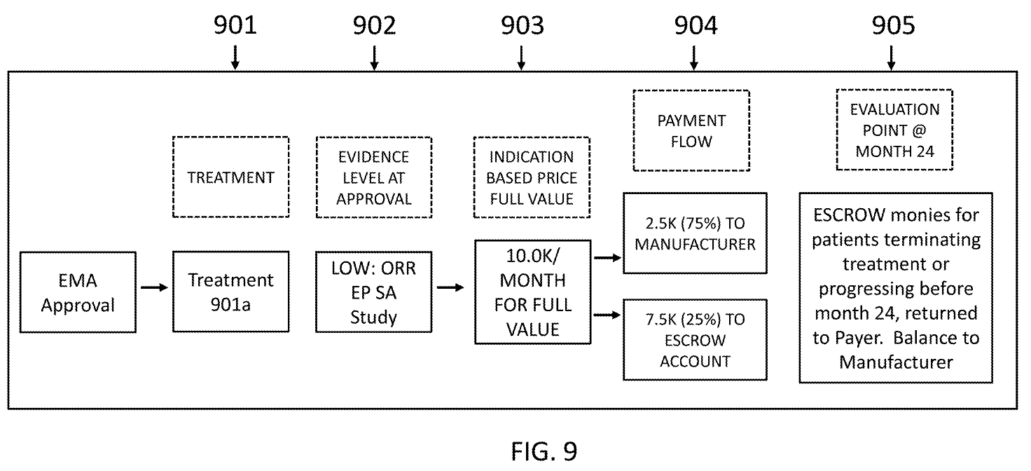

FIG. 9 shows an example of a based pricing scheme for use with an electronic record infrastructure according to an exemplary embodiment. FIG. 9 illustrates an example of a limited evidence value-based pricing scheme, which is, for example, advantageous in cases involving fast-track drug approvals, based on limited clinical trial data or real-world evidence. In these cases, payers may be put in a situation where they do not know the added benefit of the treatment at the time of negotiations with manufacturers. While it may be possible to demand a low price, it may not be sustainable and the manufacturer may decide to exit that market or fail to agree on reimbursement terms for the drug or indication if the initially negotiated price is too low. In an exemplary embodiment, the electronic record infrastructure determines the value-based price based on a risk sharing scheme using incremental evidence.

In the example shown in FIG. 9, the patient receives the treatment 901a (e.g., Opdivo® (nivolumab)) after approval from a regulator body such as the European Medicines Agency (EMA) or the Food and Drug Administration (FDA), as shown in 901. As shown in 902, the evidence level at approval by the EMA in this example is low based on an Overall Response Rate (ORR) End Point (EP) Single Arm (SA) study. As shown at 903, the electronic record infrastructure determines a valued based price for the treatment of $10,000 per month for full value (e.g., the monthly value as agreed upon by payer and manufacturer based on current and future evidence under this scheme). As shown in 904, the electronic record infrastructure determines a payment flow. In this example, the payment flow is $2,500 (25%) to the manufacturer and $7,500 (75%) to an escrow account. As shown in 905, at an evaluation point, e.g., twenty-four months in this example, the electronic record infrastructure can return the escrow money to the payer for patients terminating treatment prior to twenty-four months or progressing beyond twenty-four months of treatment. Alternatively, if the electronic record infrastructure determines the patient completed the course of treatment and did not begin an additional course of treatment, the electronic record infrastructure releases the escrow money to the manufacturer. This allows the value-based price to be adjusted based on the incremental performance of the treatment at the patient level. For example, when the electronic record infrastructure processes the patient's electronic record and determines that less than a full course of treatment was administered or that a subsequent treatment was administered, this implies that a patient had issues causing discontinuation or disease progression, respectively, and the escrow money can be returned to the payer. Conversely, if the patient completes the treatment without subsequent treatment, this implies the treatment was successful and the escrow money can be paid to the manufacturer. The example shown in FIG. 9 is exemplary. In other cases, the drugs administered, the costs, the rebates, and other factors are varied and valued based prices are determined based on any one or more of the factors discussed herein.

As described extensively above, in some embodiments, patients, payers (e.g., insurance companies), and sellers (e.g., drug companies or supplies), reach agreements on the pricing of drugs based upon diagnoses, indications, outcomes, etc. In accordance with some embodiments, the agreements can be memorialized in any suitable manner. For example, in some embodiments, the agreements can be memorialized using smart contacts. As known in the art, a smart contract is a computer implementation of a protocol to create, verify, and enforce the terms of a contract. For example, in some embodiments, a smart contract can cause a payment from one party to another to automatically be made upon agreed upon conditions being met. A smart contract can be implemented in any suitable manner in some embodiments. For example, in some embodiments, a smart contract can be written using SOLIDITY.

As also described extensively above, in some embodiments, payments can be made between patients, payers, and sellers. The accounting of these payments can be managed in any suitable manner. For example, in some embodiments, the accounting can include generating paper invoices and mailing checks. As another example, in some embodiments, the accounting can include sending electronic invoices and making electronic payments. As yet another example, in some embodiments, the accounting can include recording all transactions (amounts owed, payments, refunds, receipts, etc.) in an electronic ledger such as a blockchain ledger.

In some embodiments, updates of inputs to the mechanisms described herein, determinations of prices and amounts owed, and accounting steps can be performed at any suitable times. For example, in some embodiments, these actions can be performed in real time (or near real time), on a periodic basis, at times based on workload, or at any other suitable times.

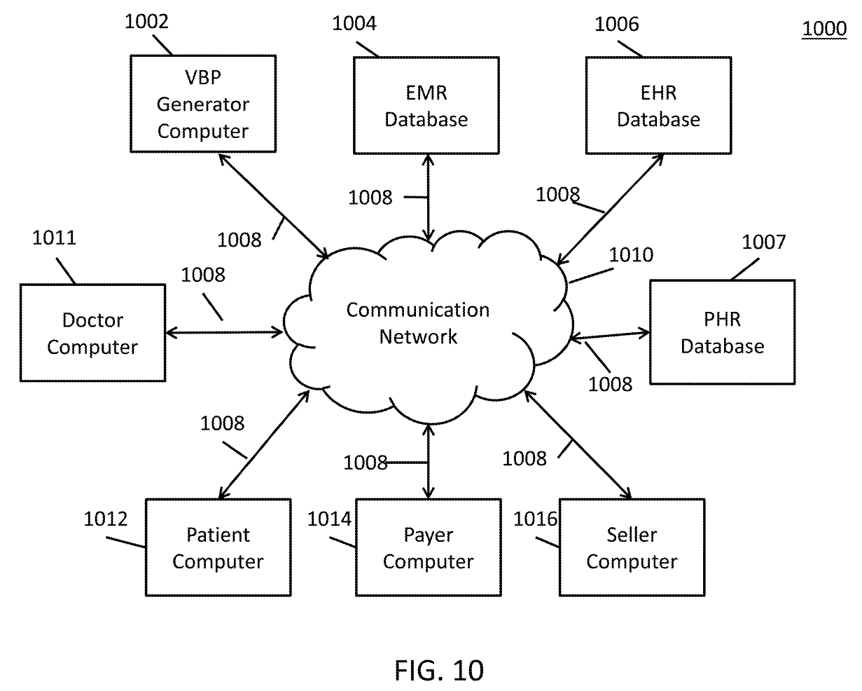

Turning to FIG. 10, an example 1000 of a block diagram of a system that can be used to implement the infrastructure described herein is shown. As illustrated, system 1000 can include a value-based price generator computer 1002, a EMR database 1004, an EHR database 1006, a PHR database 1007, communication links 1008, a communication network 1010, a doctor computer 1011, a patient computer 1012, a payer computer 1014, a seller computer 1016, and/or any other suitable components.

Value-based price generator computer 1002 can be any suitable general purpose or special purpose computer or server for determining value-based prices as described herein and/or performing any of the functions of the value-based price generator and/or value-based price generator algorithm as described herein.

EMR database 1004, EHR database 1006, and PHR database 1007 can be any suitable database, computer, or server for storing, receiving, and providing EMR, EHR, and PHR data or information.

Doctor computer 1011, patient computer 1012, payer computer 1014, and seller computer 1016 for interacting with other components of system 1000. For example, a patent computer 1012 can be a laptop computer, a desktop computer, a tablet computer, a mobile phone, etc.

In some embodiments, any of value-based price generator computer 1002, EMR database 1004, EHR database 1006, PHR database 1007, doctor computer 1011, patient computer 1012, payer computer 1014, and seller computer 1016 can be integrated with any other of these components. For example, doctor computer 1011 can be integrated with EMR database 1004. Likewise, any of value-based price generator computer 1002, EMR database 1004, EHR database 1006, PHR database 1007, doctor computer 1011, patient computer 1012, payer computer 1014, and seller computer 1016 can be implemented in any suitable number of computers.

Communication network 1010 can be any suitable computer network such as the Internet, an intranet, a wide-area network (“WAN”), a local-area network (“LAN”), a wireless network, a digital subscriber line (“DSL”) network, a frame relay network, an asynchronous transfer mode (“ATM”) network, a virtual private network (“VPN”), a satellite network, a mobile phone network, a mobile data network, a cable network, a telephone network, a fiber optic network, and/or any other suitable communication network, or any combination of any of such networks.

In some embodiments, value-based price generator computer 1002, EMR database 1004, EHR database 1006, PHR database 1007, doctor computer 1011, patient computer 1012, payer computer 1014, and seller computer 1016 can be connected to communication network 1010 through communication links 1008. In some embodiments, communication links 1008 can be any suitable communication links, such as network links, dial-up links, wireless links, hard-wired links, any other suitable communication links, or a combination of such links.

In some embodiments, communication network 1010 and communication links 1008 can be omitted when not needed.

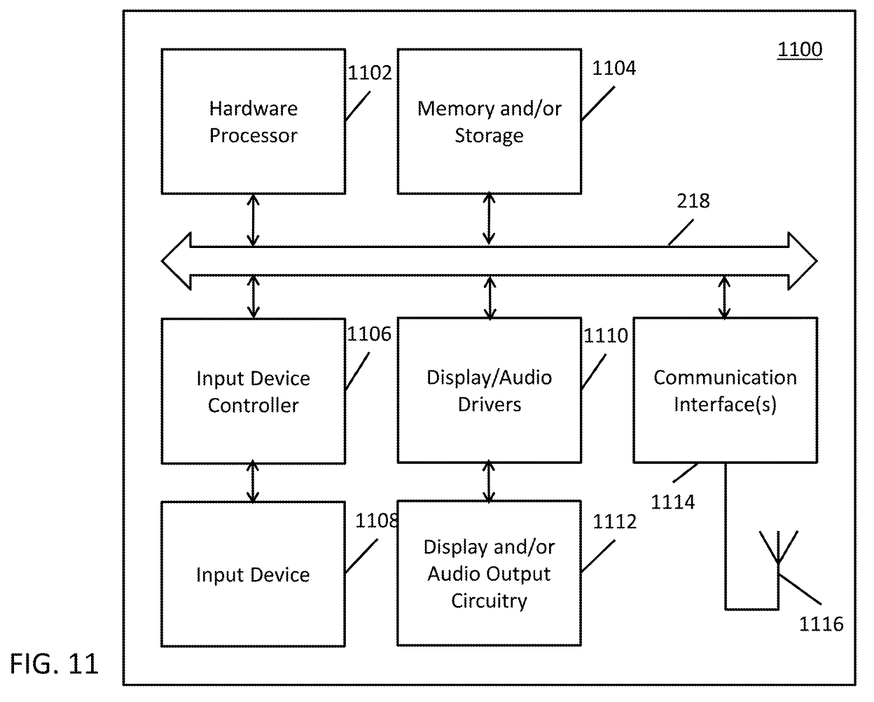

Each of value-based price generator computer 1002, EMR database 1004, EHR database 1006, PHR database 1007, doctor computer 1011, patient computer 1012, payer computer 1014, and seller computer 1016 can include and/or be any of a general-purpose device such as a computer or a special-purpose device such as a client, a server, and/or any other suitable device. Any such general-purpose computer or special-purpose computer can include any suitable hardware. For example, as illustrated in example hardware 1100 of FIG. 11, such hardware can include a hardware processor 1102, memory and/or storage 1104, an input device controller 1106, an input device 1108, display/audio drivers 1110, display and/or audio output circuitry 1112, communication interface(s) 1114, an antenna 1116, and a bus 1118.

Hardware processor 1102 can include any suitable hardware processor, such as a microprocessor, a micro-controller, digital signal processor, dedicated logic, and/or any other suitable circuitry for controlling the functioning of a general-purpose computer or special purpose computer in some embodiments.

Memory and/or storage 1104 can be any suitable memory and/or storage for storing programs, data, metrics, and/or any other suitable information in some embodiments. For example, memory and/or storage 1104 can include random access memory, read only memory, flash memory, hard disk storage, optical media, and/or any other suitable storage device.

Input device controller 1106 can be any suitable circuitry for controlling and receiving input from one or more input devices 1108 in some embodiments. For example, input device controller 1106 can be circuitry for receiving input from a touch screen, from one or more buttons, from a voice recognition circuit, from a microphone, from a camera, from an optical sensor, from an accelerometer, from a temperature sensor, from a near field sensor, and/or any other suitable circuitry for receiving user input.

Display/audio drivers 1110 can be any suitable circuitry for controlling and driving output to one or more display and/or audio output circuitries 1112 in some embodiments. For example, display/audio drivers 1110 can be circuitry for driving an LCD display, a speaker, an LED, and/or any other display/audio device.

Communication interface(s) 1114 can be any suitable circuitry for interfacing with one or more communication networks, such as communication network 1010 in some embodiments. For example, interface(s) 1114 can include network interface card circuitry, wireless communication circuitry, and/or any other suitable circuitry for interfacing with one or more communication networks.

Antenna 1116 can be any suitable one or more antennas for wirelessly communicating with a communication network in some embodiments. In some embodiments, antenna 1116 can be omitted when not needed.

Bus 1118 can be any suitable mechanism for communicating between two or more of components 1102, 1104, 1106, 1110, and 1114 in some embodiments.

Any other suitable components can be included in hardware 1100 in accordance with some embodiments.

In some embodiments, any suitable computer readable media can be used for storing instructions for performing the processes described herein. For example, in some embodiments, computer readable media can be transitory or non-transitory. For example, non-transitory computer readable media can include non-transitory media such as non-transitory magnetic media (such as hard disks, floppy disks, and/or any other suitable media), non-transitory optical media (such as compact discs, digital video discs, Blu-ray discs, and/or any other suitable optical media), non-transitory semiconductor media (such as flash memory, electrically programmable read only memory (EPROM), electrically erasable programmable read only memory (EEPROM), and/or any other suitable semiconductor media), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

The provision of the examples described herein (as well as clauses phrased as “such as,” “e.g.,” “including,” and the like) should not be interpreted as limiting the claimed subject matter to the specific examples; rather, the examples are intended to illustrate only some of many possible aspects.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims that follow. Features of the disclosed embodiments can be combined and rearranged in various ways.

What is claimed is:

1. An electronic data infrastructure comprising:
a memory storing instructions; and
a hardware processor to execute the instructions to:
identify a first electronic data record for a first patient based on a first identifier associated with the first electronic data record in a medical information electronic data repository;
analyze the first electronic data record to identify first data corresponding to patient information for the first patient based on a second identifier;
analyze the first electronic data record to identify second data corresponding to a first therapeutic intervention for the first patient based on a third identifier;
set a value for the first therapeutic intervention based at least in part on the first data and the second data; and
associate the value for the first therapeutic intervention with the first electronic data record.

2. The electronic data infrastructure of claim 1,
wherein the first data comprises an approved indication treated with the first therapeutic intervention; and
the hardware processor further to execute the instructions to determine the value for the first therapeutic intervention based at least in part on the approved indication treated with the first therapeutic intervention.

3. The electronic data infrastructure of claim 1, the hardware processor further to execute the instructions to determine the value by associating the second identifier and the third identifier with a corresponding second identifier and a corresponding third identifier associated with an agreed value table.

4. The electronic data infrastructure of claim 1, the hardware processor further to execute the instructions to
analyze the first electronic data record to identify third data corresponding to a second therapeutic intervention for the first patient based on a fourth identifier, the second therapeutic intervention is administered to the patient in combination with the first therapeutic intervention; and
determine the value for the first therapeutic intervention based at least in part on the first therapeutic intervention being administered in combination with the second therapeutic intervention.

5. The electronic data infrastructure of claim 4, wherein the first therapeutic intervention is a first drug and the second therapeutic intervention is a second drug.

6. The electronic data infrastructure of claim 4, wherein the first therapeutic intervention is a drug and the second therapeutic intervention is a non-drug intervention.

7. The electronic data infrastructure of claim 1,
wherein the first data comprises a patient's treatment results associated with administering the first therapeutic intervention; and
the hardware processor further to execute the instructions to determine the value for the first therapeutic intervention based at least in part on the treatment results associated with administering the first therapeutic intervention.

8. The electronic data infrastructure of claim 1,
wherein the first data comprises a duration the first therapeutic intervention has been administered; and
the hardware processor further to execute the instructions to determine the value for the first therapeutic intervention based at least in part on the duration the first therapeutic intervention has been administered.

9. The electronic data infrastructure of claim 1, the hardware processor further to execute the instructions to:
determine a first payer value for the first therapeutic intervention for a first payer based at least in part on the first data and the second data; and
determine a second payer value for the first therapeutic intervention for a second payer based at least in part on the first data and the second data.

10. The electronic data infrastructure of claim 1, wherein the first electronic data record is at least one of an electronic medical record, an electronic health record, or a personal health record.

11. The electronic data infrastructure of claim 1, the hardware processor further to execute the instructions to:
receive agreed pricing data from at least one of a seller and a payer;
determine the value for the first based at least in part on the agreed pricing data; and
send the value to at least one of the seller and the payer.

12. The electronic data infrastructure of claim 1, the hardware processor further to execute the instructions to determine the value and send the value to the at least one of the seller and the payer without sharing the first data with the seller or the payer.

13. The electronic data infrastructure of claim 1, wherein the first therapeutic intervention is a drug.
